(19) United States
(12) Patent Application Publication
(10) Pub. No.: US 2025/0295778 A1
(43) Pub. Date: Sep. 25, 2025

(54) MATERIALS AND METHODS FOR REPEATABLE MAGNETIC NANOPARTICLE-BASED HEATING FOR TUMOR ABLATION

(71) Applicant: REGENTS OF THE UNIVERSITY OF MINNESOTA, Minneapolis, MN (US)

(72) Inventors: Saurin KANTESARIA, Minneapolis, MN (US); Lakshya GANGWAR, Minneapolis, MN (US); Qi SHAO, Minneapolis, MN (US); Zhe GAO, Minneapolis, MN (US); Djaudat IDIYATULLIN, Minneapolis, MN (US); Zonghu HAN, Minneapolis, MN (US); Michael GARWOOD, Minneapolis, MN (US); John BISCHOF, Minneapolis, MN (US); Michael ETHERIDGE, Minneapolis, MN (US); Jacqueline PASEK-ALLEN, Minneapolis, MN (US); Bharathi JAGADEESAN, Minneapolis, MN (US)

(21) Appl. No.: 18/869,675

(22) PCT Filed: May 31, 2023
(86) PCT No.: PCT/US2023/067718

§ 371 Date: Nov. 26, 2024

Related U.S. Application Data
(60) Provisional application No. 63/347,413, filed on May 31, 2022.

(51) Int. Cl. A61K 41/00; A61B 6/00; A61B 17/12; A61B 18/00; A61B 18/04; A61B 34/10; A61K 9/00; A61K 49/04; A61K 49/18; A61L 27/18; A61P 35/00; G01R 33/28; G01R 33/48

(52) U.S. Cl. CPC A61K 41/0052; A61B 6/481; A61B 6/5247; A61B 18/04; A61B 34/10; A61K 9/0019; A61K 49/0438; A61K 49/1818; A61L 27/18; A61P 35/00; G01R 33/281; G01R 33/4812; A61B 17/12186; A61B 2018/00577; A61B 2034/104

(57) ABSTRACT

A method and a system for thermally or hyperthermally treating an object. A precipitating hydrophobic injectable liquid (PHIL) embolic agent is prepared and enhanced with a magnetic nanoparticle (NP). A delivery device is advanced to a target area and the PHIL−IONP embolic agent is injected directly at the target area. The PHIL and IONPS are observed in-situ using complementary imaging and an impulse is applied to the target area to generate heat sufficient to thermally ablate or induce hyperthymia at the target area. Additional impulses applied to the target areas at later times generate heat sufficient to ablate or induce hyperthymia at the target.

This application is being filed on May 31, 2023, as a PCT International Patent application and claims the benefit of and priority to U.S. Provisional patent application Ser. No. 63/347,413, filed May 31, 2022, the entire disclosure of which is incorporated by reference herein in its entirety.

### TECHNICAL FIELD

Embodiments relate to surgical instruments, devices or methods for transferring non-mechanical forms of energy to or from the body, in particular ablation for precise removal of tumors.

### BACKGROUND

Tumors can be treated using a variety of conventional techniques, including surgery, chemotherapy and radiation therapy. Tumors can also be reduced or eliminated by ablation typically under image guidance, such as radiofrequency ablation, microwave ablation, high intensity focused ultrasound (HIFU), or laser ablation, while other interventional oncology techniques such as embolization treat tumor by preventing the blood supply.

Initial surgical, chemotherapeutic, radiation or focal treatments may not completely remove deep seated tumors of the liver, brain and other organ systems, which often can recur even with initial success. Surgical resection can be forced to leave some tumor behind depending on location and complications. Repeating treatments is often invasive and traumatic, generally meaning secondary injections or surgeries. Non-invasive or minimally-invasive methods to treat residual or recurrent tumor are therefore very helpful to the patient. Such therapies consist of RF ablation, microwave ablation, cryoablation and various endovascular techniques.

Thermal therapy is generally either thermal ablation, which produces rapid localized heating during seconds to minutes to destroy the target tissue, or hyperthermia, which produces temperature elevation by several degrees above normal body temperature for an extended period. Magnetic fluid hyperthermia (MFH) is an emerging minimally invasive thermal therapy where magnetic nanoparticles (NPs) are injected into an area of interest and then externally heated (due to magnetic hysteresis losses) in the presence of an alternating magnetic field (AMF) at the low radiofrequency range (40-400 kHz). Low frequency electromagnetic fields have been applied for human use such as treatment for glioblastoma in the past few decades.

Because the interaction of electromagnetic fields with tissue induces eddy-current-based Joule heating, which is proportional to the frequency of these fields, the low frequency range (<400 kHz) is ideal for avoiding non-specific tissue (eddy) heating during a heating treatment. For these low frequencies there is no significant attenuation of the fields in the tissue and hence deeper penetration can be achieved, which is useful for deep tumor applications. For clinical use, homogeneity of the alternating magnetic fields is critical for ensuring spatial uniformity of power deposition in tissue as a measure of patient safety. Further, as eddy current heating also depends upon the size of the treated system, the product of magnetic field strength (H) and frequency (f) could be limited to a threshold value (such as 4 kA/m and 150 kHz for a ˜30 cm diameter based on the size of the human torso) for avoiding potential significant heating of normal tissue of a patient. MFH has several advantages over other thermal therapy delivery approaches (e.g., RF, microwave, or laser ablation), including being minimally invasive, enabling increased penetration of magnetic field in tissues, improved accumulation of magnetic NPs in tumors via magnetic targeting, and selective heating through power deposition at specific targets. This is promising for treatment of localized, vascularized, solid tumors, such as glioblastoma multiforme (GBM), hepatic tumors, etc. as minimally invasive, targeted therapy minimizes complications. The development of magnetic nanoparticles that enable quantitative imaging and predictive heating has made MFH a promising thermal therapy.

However, effective MFH treatment for vascularized solid tumors remains challenging to address, as injected NP solutions can quickly diffuse in the bloodstream or other surrounding tissues. Heating efficacy is directly related to the concentration of magnetic NPs at the targeted region, so diffusion of the NP solutions results in lower heating and a less effective treatment.

### SUMMARY

Repeatably heatable materials, and systems and methods for the placement of such materials, are described herein. Such materials can be actuated using an input (e.g., a dynamic electromagnetic energy field) that causes heating of the materials at a location of primary tumor or tumor recurrence. The materials are selected and positioned such that they can be actuated multiple times, and do not significantly diffuse or decay over time.

In one embodiment, a method for ablating or inducing hyperthermia in an object includes preparing a precipitating hydrophobic injectable liquid (PHIL) embolic agent enhanced with a magnetic nanoparticles (NP), advancing a delivery device to a target area, injecting the PHIL embolic agent directly at the target area, and applying an impulse to the target area to generate heat sufficient to ablate or induce hyperthermia in the target area. The target area is monitored for growth over weeks or months and repeated heating treatments are applied as needed, followed by possible resection after complete tumor necrosis.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is flowchart of a method for delivering a mixture used for ablation or hyperthermia according to an embodiment.

FIG. 2 is example precipitation of IONP embedded in PHIL according to an embodiment.

FIG. 3 is an example of tablet formation around a fiber optic according to an embodiment.

FIG. 4 is a 3D AVM model, where agarose 3D AVM replaced silicone.

FIGS. 5A and 5B are an example of 120 kW 15 minute heating of a model organism.

FIGS. 6A and 6B are an example PHIL/IONP Fe quantification using relaxometry.

FIGS. 7A and 7B are an example of visual evidence of IONPs in precipitate versus in solution.

FIG. 8 is an example of PHIL/IONP coprecipitates in water after 6 months.

FIG. 9A is a graph showing DLS determination of stability of IONPs in DMSO.

FIG. 9B is an image of the solutions of FIG. 9A at initial time and 24 hours.

FIGS. 10A-10F are an example of a comparison of μCT and MR imaging of PHIL-sIONP tablets.

FIG. 11A presents statistical analysis of μCT imaging using two-sample unpaired t-test.

FIG. 11B. presents statistical analysis of MR imaging using two-sample unpaired t-test.

FIGS. 12A-12D show a cross-section of the MR images of 1 mg Fe/mL tablets.

FIG. 13A is an example data set of heating of tablets in 15 kW coil at 184 kHz, and 65 kA/m demonstrating that heating of PHIL-EMG may be lower than PHIL-sIONP for the same concentrations.

FIG. 13B is an example data set of heating of tablets in 15 kW coil at 184 kHz, and 65 kA/m demonstrating that sIONPs in PHIL are able to generate more than the minimum therapeutic temperature rise (ΔT) needed for magnetic hyperthermia.

FIG. 14 is a graph of a statistical analysis of tablet heating using one-way ANOVA.

FIGS. 15A-15C are an example result demonstrated by the heating of a model organism in a 120 kW RF coil with 8 mg Fe/mL IONPs in PHIL injected in hindlimb tumor.

FIG. 16 is a graph of stability of heating in physiological conditions, 8 mg Fe/mL of sIONPs in PHIL in 1 mL PBS on an incubating shake at 37° C. and 170 RPM for 30 days.

FIG. 17A is graph showing hydrodynamic radius of IONPs in PHIL-LV-DMSO.

FIG. 17B is a graph showing hydrodynamic radius of IONPs in PHIL-DMSO.

FIGS. 18A-18C show an AVM model with a 4 mg Fe/mL PHIL-DMSO-sIONP injection and heating on 120 kW RF coil.

FIG. 19 is a graph of statistical analysis of the AVM 3D model heating of 8 mg/mL sIONPs in PHIL using one-way ANOVA.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Disclosed herein is an iron oxide nanoparticle (IONP) enhanced precipitating hydrophobic injectable liquid (PHIL) embolic as a localized dual treatment implant for dual nutrient deprivation and multiple repeatable thermal therapy of tumors. Following a single injection, multiple thermal treatments can be applied repeatedly over time as needed, based on monitoring of tumor growth or recurrence. The disclosed treatment implant provides the ability to create an injectable stable IONP PHIL solution and monitor deposition of PHIL-IONP precipitate dispersion by μCT and gauge the IONP distribution within the embolic by magnetic resonance imaging. Secondary injections can be made for increased heating or precipitate additions following tumor growth changes. Once precipitated the implant can heat to reach therapeutic temperatures (e.g., temperature elevation greater than 8° C.). Heat output may not be affected by physiological conditions, prior heating, or time elapsed between implant of the solution and initiation of a heating (e.g., up to one month). The disclosed treatment implant also provides the ability to quickly and non-invasively heat the embolic to “ablative” temperature (e.g., elevation of 17° C. in the first 5 minutes) and maintain the temperature rise over +8° C. (clinically a temperature of 45° C.) for longer than 15 minutes.

Ablation of a tumor can take place through delivery of electrical current or charge, heating, cooling, exposure to ionizing radiation or mechanical energy. Conventional techniques use these, as well as direct resection or chemical treatments, to remove or destroy tumors. It is desirable to reduce the amount of unnecessary damage to adjacent tissues during destruction of the tumor while adequately destroying tumor cells to prevent recurrence.

In some cases, it may be possible to perform a surgical operation to directly resect the tumor or growth. Tumor resection may remove all or part of the tumor, depending on physiology. Partial resection may be required to preserve the life of the patient, due to blood supply or location of the tumor. In other circumstances, however, surgery may be dangerous to the health of the patient. Even where surgery would not be particularly dangerous, it may nevertheless be preferable to treat a tumor using a minimally invasive or non-invasive treatment. Additionally a combination approach may be used, first to treat the tumor to decrease size or complications and then surgical resection when safer to complete. Two such non-invasive mechanisms for destroying a tumor include cutting off blood flow to the tumor and ablating the tumor.

One method for treatment of a tumor or other growth is to reduce or eliminate the blood flow using embolic agents. For example, embolic beads or liquid embolic agents can be delivered to an artery that is providing a blood supply to a tumor. Without the resources from the blood flow, the tumor cannot survive and will deteriorate. An embolic agent can also be directly injected into the tumor (by percutaneous or direct intra-operative visualization, for example).

Ablation can be accomplished in several ways. For example, direct radio-frequency electrical ablation can be delivered to a tumor, such an RF electrode attached to a catheter inserted to area of interest. Energy to ablate a tumor can be delivered by other mechanisms as well, such as by ultrasound. Some treatments employ radiation from an injected substance, such as microbeads of yttrium 90 that are injected at the site of the tumor. Use of these non-surgical mechanisms should nonetheless be targeted as precisely as possible.

It can be difficult to deliver radio frequency or ultrasound energy precisely to a tumor when the tumor is deep in the body or adjacent to a major blood vessel. Embolic agents can however often be delivered quite precisely, using catheters or needles to deliver the embolic to a specific location. The catheter or needle can be guided using fluoroscopy or ultrasound, for example, to target the embolic material to a very specific location. If liquid embolic material in particular is delivered to the location, it can remain solidified and remain securely in place without systemic disbursement as the vascular supply to the area has been occluded, with selection of appropriate embolic materials.

Despite these advantages of embolics and liquid embolics in particular, several drawbacks remain with magnetic embolic heating treatments in embolics that have been demonstrated previously. Onyx, a liquid embolic that has been previously combined with magnetic nanoparticles (NPs), as described in Applicant's co-pending application U.S. Ser. No. 15/912,187, is a suspension that sediments over time and therefore requires mixing immediately before injection. The water stability of hydrogels also provides a platform for the degradation of the gel in physiological systems, as it resolubilizes. Degradation of the gel matrix will result in loss of IONPs and is further exacerbated by heat from RF treatments. One week of time is typically needed to observe if tumors are growing after a clinical MFH treatment; if heating is not consistent over a week (i.e., the NPs must not degrade or diffuse from the implant site), repeat treatment cannot be tailored to the patient. Thus, there is a present need in the art to improve the combination of MFH with embolization for multiple heating regimes tailored to the growth of the tumor.

Disclosed herein is a shelf stable liquid embolic that acts as an implant, to provide consistent and predictable heating, which is not degraded by physiological conditions or magnetic embolic heating. Precipitating hydrophobic injectable liquid (PHIL) is a liquid embolic agent, that is combined with magnetic NPs for this purpose. In embodiments, PHIL is composed of a nonadhesive copolymer (polylactide-co-glycolide (PLGA) and polyhydroxyethylmethacrylate (PHEMA)) dissolved in DMSO with an iodine component (triiodophenol) covalently bound to the copolymer, causing radiopacity. The precipitated hydrophobic injectable polymer, iodinated PLGA-PHEMA polymer (referred to herein as PHIL), when in solution will be referred to as PHIL-DMSO. As PHIL is not soluble in water, so once precipitated in physiological conditions it provides long term stability as a framework to support magnetic NPs. Magnetic NPs need to be colloidally stable in PHIL-DMSO, and additionally have the capacity to generate high heating and are locatable using MRI imaging. IONPs are often used for heating, and their coatings significantly affect their stability in various solutions. EMG308 (referred to as EMG) and silica coated EMG, sIONP have been tested in DMSO containing solutions where EMG is not stable but sIONPs are. The present disclosure provides for future minimally invasive vascularized tumor treatment by injection of a liquid embolic PHIL and iron oxide nanoparticle (IONP) mixture in combination with magnetic embolic heating.

The ability to concentrate or prevent loss of magnetic NPs or the heating efficiency at the area of interest is promising, particularly since it would not lengthen injection time. Previous work has showed that adding targeting functionalities to magnetic NPs coating still results in low accumulation in the area of interest. A potential way to increase local delivery of magnetic NPs and decrease their diffusion from the target site is to combine them with a non-diffusing carrier such as a liquid embolic agent. Liquid embolic agents, liquid injection that gels or solidifies in-situ such as hydrogels or thermo polymers, have been used extensively in the past for treatment of arteriovenous malformations (AVMs) and more recently for treatment of tumors. They work by reducing blood supply to the tumor and producing ischemia. When combined with magnetic NPs, they can be used to deliver a high local concentration of magnetic NPs to a tumor vascular bed or interstitium. These magnetic NPs can then be used to achieve local hyperthermia or ablation. The combination of embolization (with the liquid embolic) and thermal therapy (with the magnetic NPs) can allow for heating tumor beds prior to resection of the tumor or the heating tumor margins following resection of the tumor. Multiple heat treatments based on long term tumor monitoring will ideally prevent tumor recurrence at the margins of resection cavities.

Iron-oxide nanoparticles (IONPs) in embolic agents for magnetic embolic heating treatments have been explored in recent years. Alginate hydrogels have been proposed for localizing the incorporated magnetic microparticles. Furthermore injectable, biodegradable, thermosensitive and superparamagnetic iron oxide nanoparticle-loaded nanocapsule hydrogels have been demonstrated with multiple MFH and long-term magnetic resonance imaging (MRI) contrast approaches. In previously published literature, embedding magnetic NPs in liquid embolics has only been shown to preserve magnetic NPs heating for up to 2 weeks. Additionally, multiple heat treatments have been shown to decrease tumor growth.

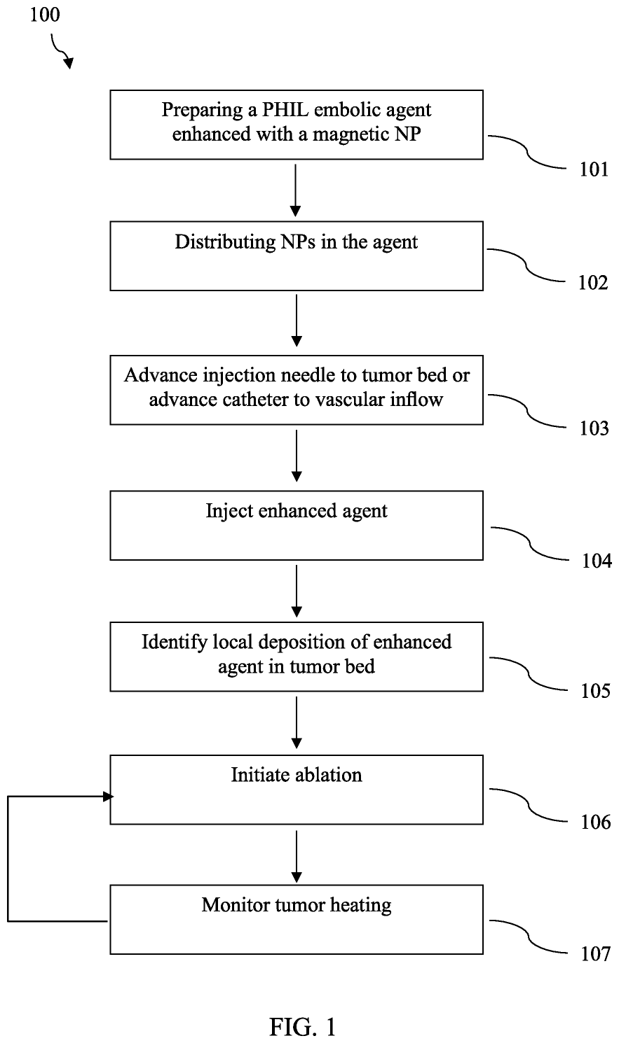

FIG. 1 is a flowchart of a method 100 according to an embodiment in which a IONP and embolic mixture is delivered to a tumor.

At 101, an admixture of a PHIL liquid embolic agent and magnetic NPs is prepared. In other embodiments, additional materials could be added to the mixture, such as coagulants, solvents, or other materials.

At 102, the magnetic NPs are suspended in the mixture uniformly. Various techniques can be used to achieve this uniform suspension. In some examples, mechanical agitation (e.g., shaking) can be used to create this uniform suspension. In some examples, the user would transfer the contents between a pre-provided embolic syringe and an empty syringe, back and forth, in order to create a uniform suspension. In other examples, a centrifuge or vortex agitator is used. In one example, once the step is taken to uniformly suspend the magnetic NPs in the agent, a needle is used to draw the enhanced agent into a separate syringe; the contents of this separate syringe are then injected into the patient.

In one particular example, a pre-filled syringe sold to the end user would include PHIL® in the form of the biocompatible polymer suspended in DMSO and separate magnetic NPs all included in the pre-filled syringe. The user would shake or use a vortex agitator to agitate the syringe so that the magnetic NPs are suspended in the syringe, and the contents of this syringe would then be transferred to another delivery syringe via a needle. Alternatively, the pre-filled syringe could contain multiple chambers which could be mixed by passing through a static mixing nozzle while transferring to the delivery syringe. This delivery syringe could then be used to deliver the enhanced agent to the tumor or other target.

At 103, a delivery device such as a needle or catheter configured to deliver the enhanced agent is advanced to the tumor bed. In embodiments, a needle is advanced to the tumor bed, and alternatively a catheter can be routed through the vasculature to a vein or artery at a vascular inflow/outflow to a tumor.

At 104, the enhanced agent is delivered to the tumor or tumor bed by the needle or catheter from 103. In embodiments, the enhanced agent can be delivered to a region or multiple insertion points, rather than a single location. In embodiments, fluoroscopy or ultrasound can be used to determine the precise position of the needle during advancement or injection at 103 and 104. The enhanced agent is injected to areas which are to be treated, such as a tumor. During and after injection, the local deposition of the mixture in the tumor bed can be identified, at 105. In embodiments, the magnetic NPs that have been injected can be used to identify the local depositions, based on feedback produced by the magnetic NPs in response to fluoroscopy (CT injection monitoring), CT, MRI, ultrasound, electrical or mechanical stimuli.

At 106, the magnetic NPs are targeted to generate heat (by application of an impulse, such as RF alternating magnetic field). Because the enhanced agent including an embolic is injected at the tumor bed or other areas which are desirably treated, there is little or no spread of the magnetic NPs to other areas. In this way, an RF field can be delivered that is not sufficient to damage tissue, but will cause temperature increase only in the region where the enhanced agent has been delivered.

At 107, tumor heating is monitored. When sufficient heating occurs at the site of the tumor or other object, a medical professional can determine that the object has been thermally treated. Thermal treatment can be repeatedly initiated based on observed character of the tumor or other target during the monitoring.

Tumor growth is monitored and if growth is observed repeated treatments are used as needed weeks to months later.

### Example 1: Preparation of IONPs with PHIL in DMSO Solutions

Seven example solutions of PHIL-DMSO with IONPs were made following formulations in Table 1.

TABLE 1
Sample formulation of IONPs in PHIL

| | Conc | Mass (g) | | | PHIL |
|---|---|---|---|---|---|
| IONP | mg Fe/mL | IONP | PHIL | DMSO | wt/vol % |
| None | 0 | 0 | 3 | 9 | 31% |
| sIONP | 1 | 0.204 | 3 | 8.96 | |
| | 2 | 0.407 | | 8.91 | |
| | 4 | 0.814 | | 8.83 | |
| EMG308 | 1 | 0.02 | 3 | 9 | |
| | 2 | 0.041 | | 8.99 | |
| | 4 | 0.081 | | 8.98 | |

Powdered PHIL (25 g) was added to DMSO (66.667 g) and heated to 60° C. for approximately 30 minutes with intermittent shaking until PHIL was completely dissolved and was used as a PHIL-DMSO stock solution. sIONPs (1.63 g) were added to DMSO (1.66 g) and point sonicated for 15 minutes at room temperature. EMG (0.081 g) were added to DMSO (1.96 g) and point sonicated for 15 minutes at room temperature. PHIL-DMSO solution (11 g) was added to each of 7 vials, sIONP-DMSO solution was added to appropriate vials (1: 0.411 g, 2: 0.822 g, 4: 1.644 g) and EMG-DMSO solution was added to appropriate vials (1: 0.265 g, 2: 0.530 g, 4: 1.061 g). Remaining DMSO was then added (0: 1 g, sIONP 1: 0.753 g, sIONP 2: 0.495, EMG 1 0.755 g, EMG 2: 0.501 g). Solutions were vortexed to mix.

### Example 2: Stability of IONPs in Solutions

Samples for dynamic light scattering (DLS) characterization of colloidal stability were made by adding PHIL-DMSO stock solution (1.393 g) DMSO (0.127 g) and either sIONP-1 or EMG-1 (0.48 g) solutions, to make 0.1 mg Fe/mL solutions. The samples were visually observed and measured on the DLS using a standard method for measuring hydrodynamic diameter over 10 days as previously reported. Nanoparticle size was determined by DLS, measurements on a Brookhaven Zeta PALS instrument (Brookhaven Instruments Corporation) with a 635 nm diode laser at 15 mW of power. Stability of IONP colloidal suspension was determined by DLS time points at the above concentration, taken several times in the first 24 hours and then on daily or weekly intervals up to 10 days. Measurements were stopped when visual precipitation of IONPs occurred. Colloidal stability of IONPs were tested in DMSO and PHIL-DMSO solutions.

### Example 3: Tablet Formation and Imaging Sample Preparation

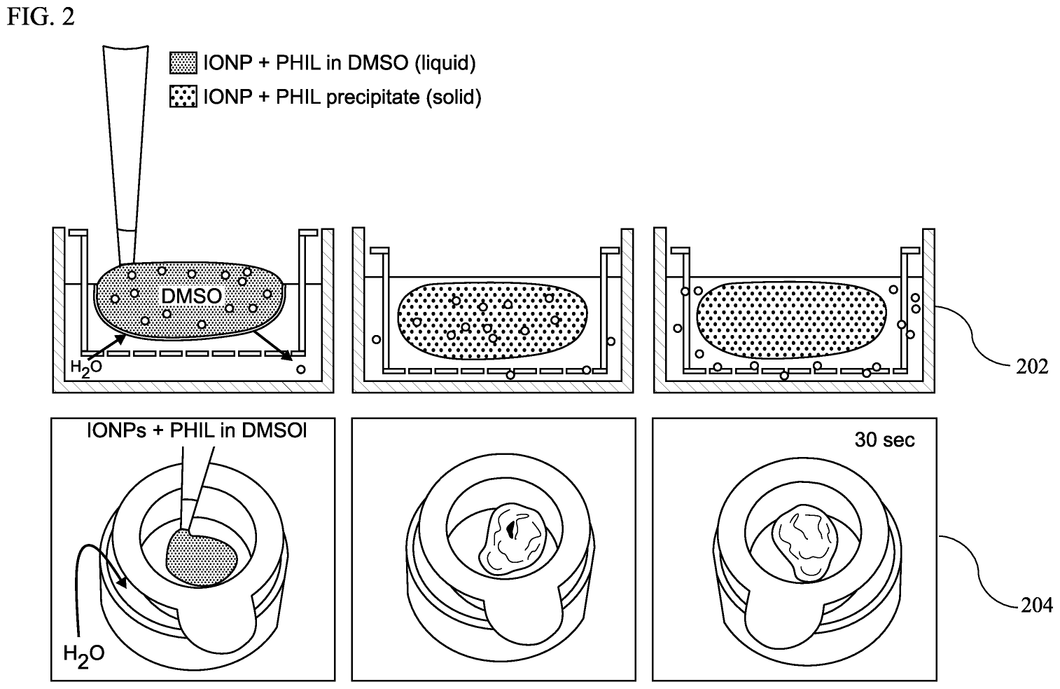

A mesh cell strainer was placed in a jar lid and surrounded with approximately 0.5 cm of deionized (DI) water. PHIL-DMSO and PHIL-DMSO-IONPs (1, 2 and 4 mg Fe/mL solutions) solutions were pipetted gently into the water to make an approximately 1.5 cm (0.3 mL solution) diameter precipitate disk (FIG. 2). Once the exterior shell formed, after approximately 30 seconds, additional DI water was gently pipetted around the mesh filter to cover the precipitate disks. Once well formed, the PHIL tablet disk was transferred using the mesh filter to a 125 mm diameter crystalizing dish filled with 1 inch of fresh DI water (200 mL). DI water was refreshed 3 times over 4 hours, before leaving the samples overnight.

MR and microCT imaging were completed on the same tablet samples. Samples were placed in a 2 cm diameter NMR tube, layered with Teflon spacers to ensure tablets were in the MR coil measurement area. Tablets were placed layered with Teflon spacers for separation and tubes were filled with fresh DI water before running both MR and μCT imaging.

Referring now to FIG. 2, precipitation of PHIL with IONPs embedded is illustrated by diagram 202 of precipitation of PHIL-IONP tablet in water and photo time-lapse 204 of PHIL-IONP precipitation. On contact with water, DMSO contacting water instantaneously diffuses out from the PHIL-IONP solution and exchanges with water, resulting in PHIL precipitation, due to PHIL's insolubility in water. The edge of injection solution contacting water first has the fastest exchange of DMSO creating a highly porous shell. Desired shape is made by quickly extruding solution and an enlarging initial shell. Once the final structure is formed, DMSO continues to diffuse outwards from center of the precipitate. Fresh water is replaced multiple times over the first four hours and samples are left overnight in 200 mL of distilled water to ensure all DMSO is removed. The surrounding solution remains clear indicating minimal to no leakage of IONPs.

### Example 4: MicroCT Imaging of PHIL-IONP Tablets

Samples were scanned in a microCT imaging system (e.g., NIKON XT H 225, Nikon Metrology, MI). The accelerating voltage was set 121 kV, and the current was set to 150 μA. The resolution was 0.053 mm. A 1-mm aluminum filter was placed between the source and the object to reduce the beam hardening effect. The images were reconstructed to reduce the beam hardening effect by software and improve image quality (e.g., 3D CT pro, Nikon Metrology, MI). The images were then imported as unsigned 16-bit float images, post-processed (e.g., VGstudio Max 3.2, Volume Graphics, NC), and exported as DICOM images for a final analysis using MATLAB (MathWorks). The grayscales values were transferred into HU based on the air and water samples.

### Example 5: MR Imaging of PHIL-IONP Tablets

MR imaging was performed on a 16.4-T, 26-cm bore magnet (Magnex Scientific, Yarnton, UK) interfaced to a research spectrometer (Varian, Palo Alto, CA). A single-loop surface coil (diameter=2.5 cm) was used for RF transmission and signal detection. The pulse sequence was multi-band sweep imaging with Fourier transform (MB-SWIFT) combined with a Look-Locker acquisition scheme designed to measure the longitudinal relaxation time (T₁) of rapidly decaying water signals. Parameter settings were: acquisition bandwidth (BW)=500 kHz, repetition time (TR)=1.32 ms, longitudinal recovery delay (τ)=97.154 ms-2857.154 ms (16 linearly spaced points), flip angle=1°, RF pulse length=2.0 μs, acquisition delay=1.7 μs, gaps=2, and total acquisition time of −14 min. The number of views (Nᵥ) and number of complex points (Nₛₚᵢᵣₐₗ) were adjusted between Nₛₚᵢᵣₐₗ=440 or 400 and Nᵥ=100 or 80, respectively, depending on the image size. The field of view (FOV) varied from 30-40 mm in x, y, and z depending on the sample size with a resolution of 256×256×256 pixels. MB-SWIFT images were reconstructed using a custom C++ program and VnmrJ version 3.2.

### Example 6: Image Analysis

A MATLAB (MathWorks) script was used to determine both the mean T₁ and Houndsfield Units (HU) of each tablet. To determine the relaxation rate constant R₁ (1/T₁) and HUs of a given PHIL tablet, a circular region of interest (ROI) was manually selected in the approximate middle slice of a given tablet for the T₁ map and microCT image stacks. The mean T₁ and HUs were taken for this ROI, ignoring T₁ values<0 s and >1.5 s and removing outliers more than 3 median absolute deviations from the mean. The T₁ values were then converted to R₁ values by taking the reciprocal.

A separate MATLAB (MathWorks) script was used to determine the mean R₁ for the center cross section of the 1 mg Fe/mL tablets as a function of distance from the closest edge of the tablet to approximate distribution of IONPs within the tablet. A cross-section without any visible artifacts or bubbles was taken from the center of each 1 mg Fe/mL PHIL-sIONP tablet T₁ map, which again was converted to R₁ values by taking the reciprocal of the T₁ value at a given pixel. The outline of this tablet was manually drawn using ImageJ and the entire image was converted to a binary image via thresholding for nonzero pixels. The distance transform of this was then computed for the inverse of this image and the mean R₁ as a function of distance from the edge of the tablet in bands of 5 pixels (0.586 mm) was then plotted as a moving average from the edge of the tablet towards the center. ImageJ was also used to create a 3D surface plot of a representative cross section of one of these PHIL-sIONP tablets from the manually outlined MR image.

### Example 7: Heating Characterization of IONPs in PHIL Tablet

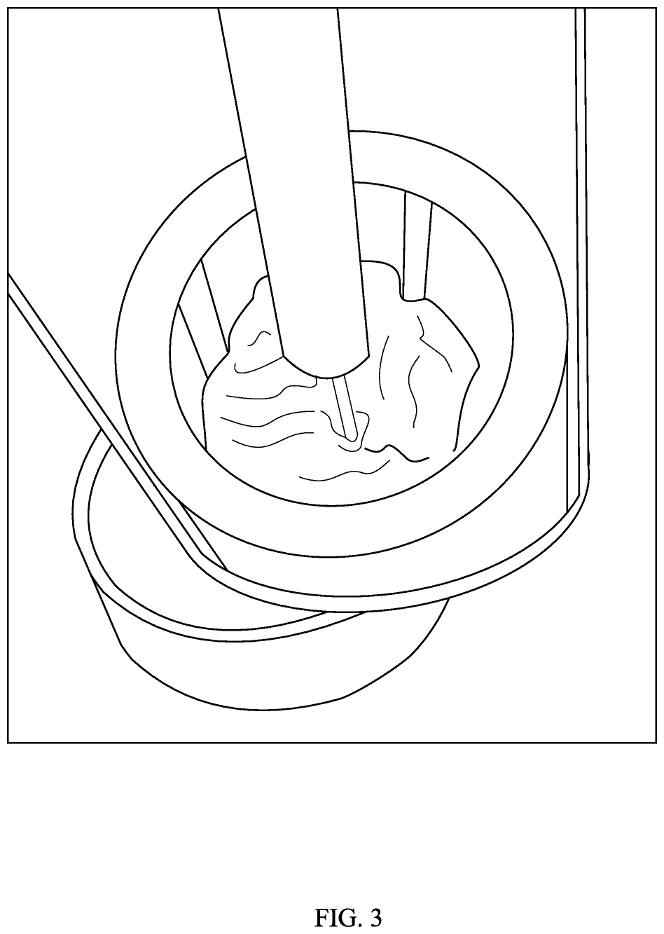

Heating experiments were performed in a 15 kW RF system on a PHIL-IONP tablet precipitated using the same method as for the imaging (Examples 4 and 5) with the addition of a fiber optic temperature probe, as shown in FIG. 3. Following immersion in DI water overnight, tablets attached to fiber optic probes were placed into a container with fresh DI water (approximately 5 mL) before the heating run. PHIL-IONP co-precipitate tablets were heated at 60 kA/m and 175 kHz using the custom-built RF coil with 5 cm ID (˜80 mL capacity) (Fluxtrol, Auburn Hills, MI). Each sample was heated 3 times for a time span of 60 seconds. A fiber optic temperature probe (Qualitrol, Fairport, NY) was used to collect the temperature inside the system. Iron content was later quantified using inductively coupled plasma mass spectrometry (ICP-MS) or relaxometry. The thermometry data was later analyzed to calculate temperature rise (ΔT=T_{final}−Tᵢₙᵢₜᵢₐₗ) as well as the volumetric specific absorption rate (SAR_{V}) which is the power deposited per unit volume (W/m³) by using the combined heat capacity of the water & PHIL tablet along with the initial slope, e.g., first 30 seconds of temperature rise recorded using the time rise method.

### Representative Calculations for SAR_{V} (or Power) Deposition:

The total power deposited (P, Watts) into an embolic tablet can be estimated as below:

$$P = {m}^{′} * {C}_{P}^{′} ⋆ \frac{Δ T}{Δ t}$$

m′ (kg): mass of combined PHIL+water tablet & C_{P}′ (J/kg·K): specific heat at constant pressure for combined PHIL+water tablet
C_{P}′: Combined specific heat of water+PHIL (tablet) is estimated by weighted mass average—

$${C}_{P}^{′} = ( {m}_{water} ⋆ {C}_{P} \text{?} + {m}_{PHIL} ⋆ {C}_{{P}_{PHIL}} ) / ( {m}_{water} + {m}_{PHIL} )$$

$$\text{?} \text{indicates text missing or illegible when filed}$$

Further, assuming the SAR_{V} is the power deposited per unit volume (W/m³) as calculated by time rise method:

$${SAR}_{V} = \frac{P}{V} = {ρ}^{′} ⋆ {C}_{P}^{′} ⋆ \frac{Δ T}{Δ t}$$

ρ′ (kg/m³): density of tablet is a combination of PHIL and water, with the vast majority of it being water. Thus the density of water (1000 kg/m³) can be used as an approximation for the density of the tablet.

To assess SAR_{V} by the time rise method, the ΔT/At slope of temperature curve over initial 30 seconds may be used. For instance, in the case of sIONP trial 5C at 5 mg Fe/mL, slope of heating curve was observed to be 0.228 K/s (average of 3 repeats). C_{P} of water=4180 J/kgK and C_{P} of PHIL=1400 J/kgK

$${C}_{P}^{′} = \frac{7.36 ⋆ 4.18 + 0.339 ⋆ 1.4}{0.339 + 7.36}$$

$${C}_{P}^{′} = 4057 [ \frac{J}{kg K} ]$$

$$SA {R}_{V} = 1000 [ \frac{kg}{{m}^{3}} ] ⋆ 4057 [ \frac{J}{kg K} ] ⋆ 0.228 [ \frac{K}{s} ]$$

$$SA {R}_{V} = 9.25 ⋆ {10}^{5} [ \frac{W}{{m}^{3}} ]$$

### Example 8: Heating Characterization of PHIL-sIONP in an Agarose 3D AVM Model

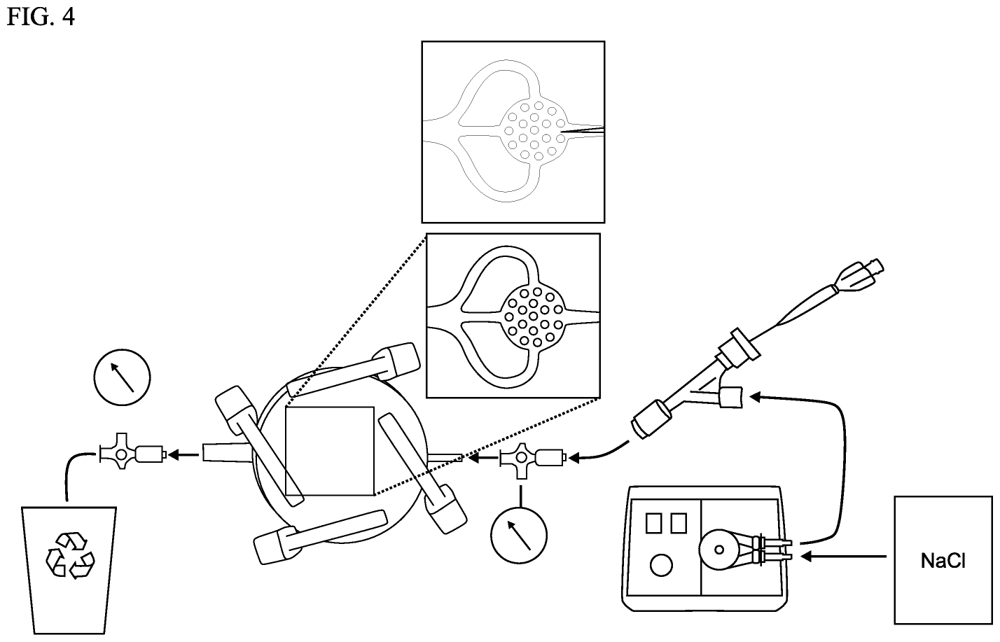

FIG. 4 is a PHIL-sIONP coprecipitation in an agarose 3D AVM model designed from a silicone model (e.g., MicroVention). The 3D model consists of an afferent tube, representing the feeding artery, supplying the artificial nidus; a round, flat, honeycomb-like 3D space; and 3 efferent tubes, representing 3 draining veins. The silicone of the provided model inductively couples to the coil and thus was unsuitable for heating runs. Therefore, a clay reverse imprint of the AVM model was recreated to have dimensions of 2 inches diameter and 1 cm thickness. 3% Agarose (3 g) in water (97 mL) solution was made by heating the mixture until completely dissolved and then immediately pouring into the clay imprint model. Once cooled, the agarose model was removed from the mold, attached to a water pump flowing at 1 mL/min, with 3-way connecter attached 1 inch from the drain inlet, and clamped between two pieces of 1 cm plexi-glass. After equilibrating the 3D agarose model with water, 0.5 mL of 4 mg Fe/mL PHIL-DMSO with IONPs was injected into the center of the honey comb area of the model. This was completed under continuous water flow, following the previously established procedure. The sample was allowed to cure for 20 minutes at 1 mL/min of water flow, and then removed from the plexi-glass set up. The agarose model was then attached to a 3D printed holder and placed inside another custom built 120 kW (at 365 kHz) RF Coil with maximum field strength of ˜35.2 KA/m (Fluxtrol, Auburn Hills, MI). Three trials of heating for 60 seconds were performed for each sample. An Infrared camera (e.g., FLIR A300 Thermal Imaging Camera, Teledyne FLIR LLC, Oregon) was used to record the temperature inside the system and the data collection is procured using the ThermaCam Researcher Pro Software. The thermometry data was analyzed by evaluating temperature rise over 60 seconds at three locations on the agarose model face. The amount of iron in the co-precipitate was later quantified using relaxometry.

### Example 9: Heating Characterization for Long Term Repeatability in Physiological Conditions

Heating data was analyzed in a similar way as previously described using a 1 kW (RF coil) Hotshot inductive heating systems with 2.75-turns, water-cooled copper coil (e.g., Ameritherm Inc., Scottsville, NY). A volume of 1 mL PBS solution was placed in a 2 mL cryotube with a blunt nose needle in the center touching the bottom. The needle acts as both placeholder for the future temperature probe and to inject water into the bottom of the tube to force DMSO up while precipitate PHIL. 1 mL of PHIL-DMSO or 8 mg Fe/mL PHIL-DMSO-sIONPs solution was injected into the cryotube through PBS. The precipitate formed around the needle inside the cryotube. To completely remove the DMSO the solution surrounding the precipitate was removed and replaced by injecting 15 mLs PBS through the needle and allowing excess solution to flow out over the top several times over 1 hour. Purification solution was removed after 1 hour, 1 mL of fresh PBS was added, and the sample was weighed. The cryotube was capped with a rubber stopper along with the fiber optic temperature probe through the center in the hole created by the needle. Temperature data was recorded in 1-second intervals for a total period of 120 seconds where the initial 30 seconds are kept for temperature stabilization, the subsequent 60 seconds for heating when the RF coil is on, and the last 30 seconds are for idle after the coil is turned off. PBS was used as a reference for coil heating characterization results. SAR_{V} (power deposited per unit volume) in the sample is measured using a 1 kW RF coil at 360 kHz and 20 kA/m and the calculation are performed in same way as previously described for PHIL tablet heating (W/m³). Each sample was tested for 3 trials at a given time point. Samples were tested at 1 hour, 24 hours, 7 days and 30 days after initial precipitation. When not being heated, samples were placed on an incubating shaker at 37° C. rotating at 170 RPM for the entirety of the experiment. PBS was replaced every 3-4 days. PBS was refreshed with new mass recorded before every heating test.

### Example 10: Heat Characterization in a Post Mortem Tumor Model Heating for 15 Minutes

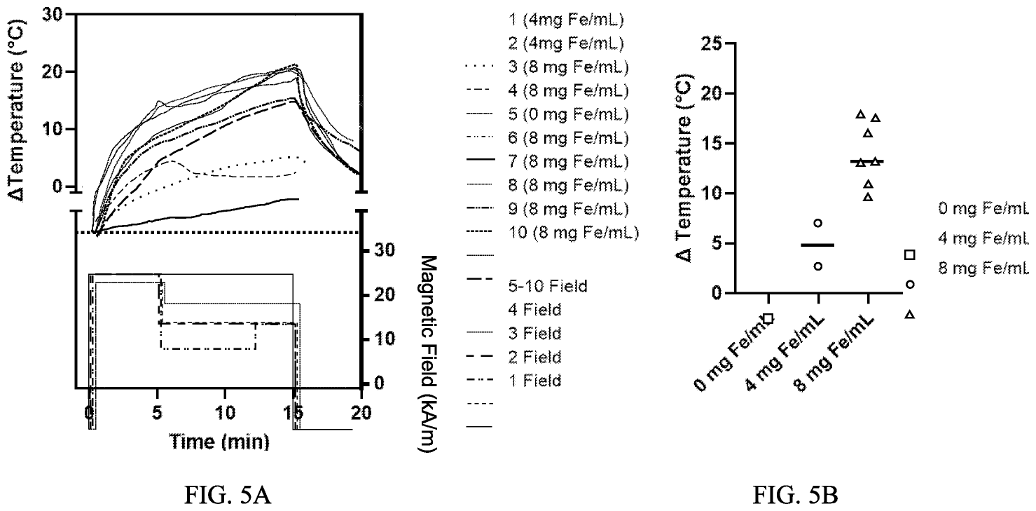

Mice bearing MC-38 tumor (model described in Ranjbartehrani et al., Characterization of Miniature Probes for Cryosurgery, Thermal Ablation, and Irreversible Electroporation on Small Animals, Advanced Therapeutics 2022) on the hind limb were humanely euthanized, in accordance with protocols approved by the Institutional Animal Care and Use Committee (IACUC), University of Minnesota. Post mortem, tumor-bearing mice were used to test the feasibility of heating to the hyperthermic range for 15 minutes in tissue. Mice were kept at 4° C. until day of use, then equilibrated to room temperature. Hind limb tumors were injected with 0.2 mL of PHIL-DMSO-IONPs (0, 4, 8 mg Fe/mL) over a time span of 3 minutes. The injection site was then flushed with 10 mL of water 4 times over 20 minutes to remove all excess DMSO. Temperature monitoring was performed with the help of an infrared camera which recorded the temperature of the tumor and the data collection was procured and analyzed using the ThermaCam Researcher Pro Software. Mice were heated in a 120 kW RF coil operating at 365 kHz with varying magnetic field strength 18-35.2 kA/m in a time span of 15 minutes as shown in, FIG. 5.

FIGS. 5A and 5B are an example of 120 kW 15 minute heating of a model organism, e.g., mice. The top portion of FIG. 5A shows temperature curves of mice injected with 0.2 mL of various concentrations of sIONPs in PHIL DMSO solution in a coil at 365 kHz and various field strengths. The bottom of FIG. 5A shows repeat of magnetic field strengths of heating trials. FIG. 5B shows temperature increase in 0-5 minutes at various concentrations of IONPs in 0.2 mL PHIL DMSO injections, red: 0 mg Fe/mL, Blue: 4 mg Fe/mL, Black 8 mg Fe/mL. Mouse number to magnetic field strength for legend.

### Example 11: Iron (Fe) Quantification

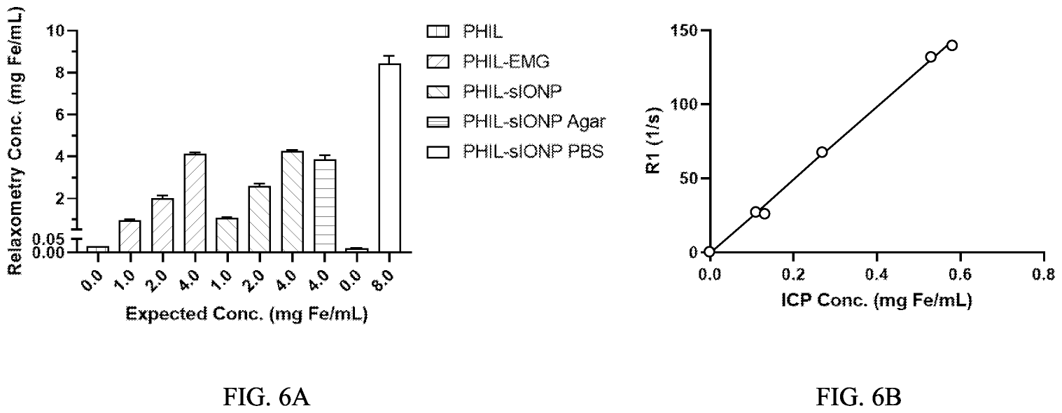

Elemental analysis by inductively coupled plasma mass spectroscopy (ICP-MS) on an Agilent 7700 was performed by Australian Laboratory Services (ALS) Global Environmental (Kelson, WA). Prior to analysis by ICP, PHIL and PHIL-IONPs tablets were lyophilized in a FreeZone 6L Console Freeze Dry System (e.g., Labconco) overnight and crushed into powder. Powder (approximately 20 mg) was digested with 0.2 mL DI water, and 0.4 mL concentrated nitric acid were flame sealed in a glass ampule and left at 100° C. overnight. Relaxometry (e.g., magnetic relaxometer, MQ60 TD-NMR ANALYZER VT, Bruker Biospin Corporation, MA) was used for further iron quantification. A linear regression of R₁ vs concentration of Fe obtained by ICP of the digested IONPs was determined from a range of concentrations (0, 1, 2, 4 mg Fe/mL powder aliquots) as shown in FIGS. 6A and 6B.

FIGS. 6A and 6B are an example PHIL/IONP Fe quantification using relaxometry. FIG. 6A shows lyophilized samples of PHIL+IONP precipitate that were crushed and digested for relaxometry and ICP Fe quantification. Concentration of injections by R1 of digested powered, normalized to amount (approximately 15-30 mg) of powdered sample digested. FIG. 6B shows linear regression of ICP reported Fe concentration to R1 values. One may note that the R1 values obtained via MR Imaging are noticeably lower than expected as IONPs can increase R1 to ˜40 l/s or so on imaging. This effect is most likely a result of the PHIL around the IONPs well as silica coating of sIONPs, limiting the amount of free water close to the IONPs compared to Fe after IONPs are digested.

### Example 12: Precipitation of IONPs in PHIL

The tablet shape, approximately 1 cm circular disk, was chosen for repeatable testing and to fit into a 2 cm NMR tube when comparing imaging and heating properties. PHIL-DMSO or PHIL-DMSO-IONPs was pipetted into a bed of water (dark colored liquid, FIG. 2, far left) where the initial contacting edge precipitates first, creating a porous shell as the bottom of the tablet shape (tan colored solid, FIG. 2). After the bottom shell forms, continued addition of PHIL-DMSO or PHIL-DMSO-IONPs may back fill porous areas rather than create new edge interface with water. Therefore, continued pipetting was directed in and around the shell to achieve the correct dimensions, the cell filter helped to control height of tablet. Additional water was added after injection to complete shell formation on the top of the tablet (tan shell covering dark liquid center FIG. 2, middle). The cell filter was used to disturb the solution, increasing diffusion of DMSO and water, to create a stable framework precipitation. Once the tablet was formed water was then refreshed multiple times to hasten diffusion of DMSO from the center of the precipitate outward (tablet visually tan throughout, dark center no longer visible, FIG. 2 right). Remaining DMSO was expected to be removed by the final steps of submersion in large volumes of fresh DI water overnight (tablet color does not change from FIG. 2, right).

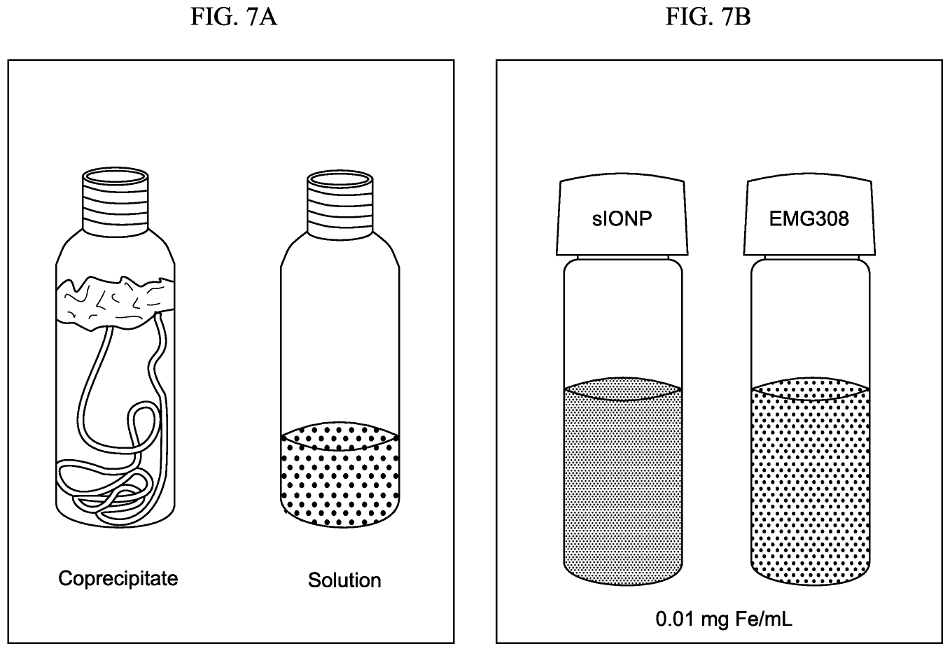

IONPs stay within the precipitate rather than pass into aqueous solution with DMSO. The IONPs embed in the precipitate as the PHIL-DMSO-IONPs are extruded into water. FIGS. 7A and 7B are an example of visual evidence of IONPs in precipitate versus in solution. In the photo image of FIG. 7A, on the right is 1 mg Fe/mL sIONP with PHIL dissolved in DMSO solution and on the left is solution pipetted into water resulting in precipitate as DMSO exchanges with water. Color of surrounding solution does not change color, IONPs are not leaking into H2O substantially. In the photo image of FIG. 7B, low concentration of IONPs in DMSO solution is shown. The color of the surrounding solution does not change, a slight brown tint can be seen even at very low concentrations, 0.1 mg Fe/mL of IONPs in solution.

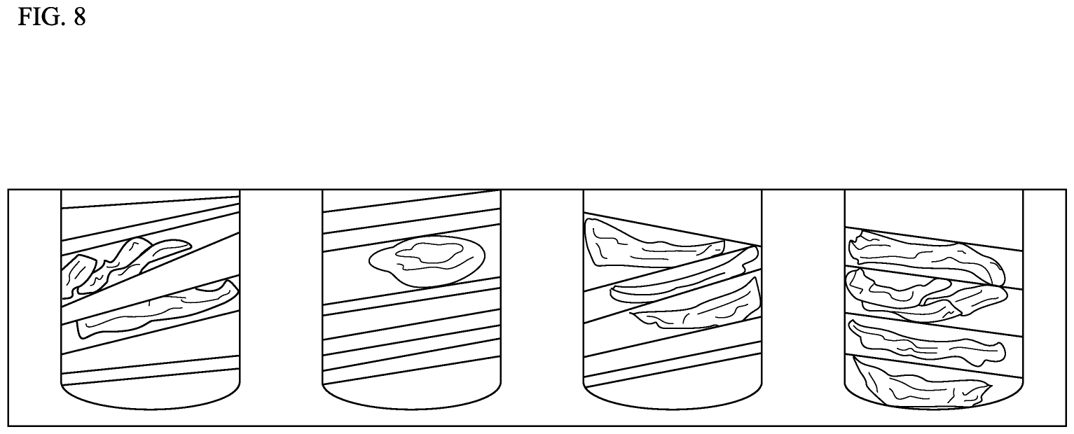

Visually all solutions surrounding precipitate, with or without IONPs, are clear in color, indicating IONPs are not diffusing out of the PHIL precipitate matrix substantially. No color change was seen in samples left in water for over 6 months, increasing confidence that IONPs are not diffusing from the tablet. FIG. 8 is an example of PHIL-IONP coprecipitates in water after 6 months. PHIL without IONPs is white, increasing concentration of sIONP results in a darker tan color, and increasing concentration of EMG308 results in a darker grey color. Fragmentation of precipitate is due to sample transport knocking tablets against glass. Tablets with more sIONPs are less fragmented. Solution remains clear indicating IONPs are not diffusing out of the tablet.

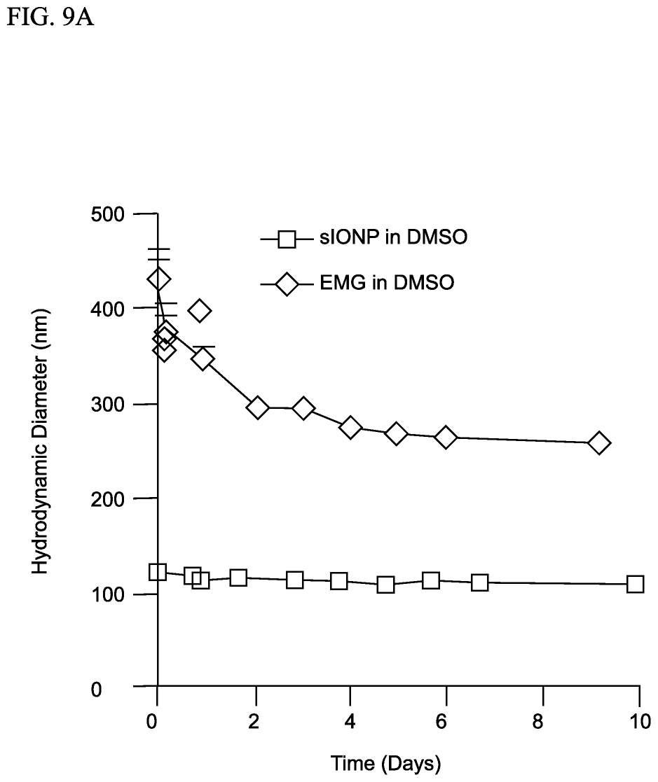

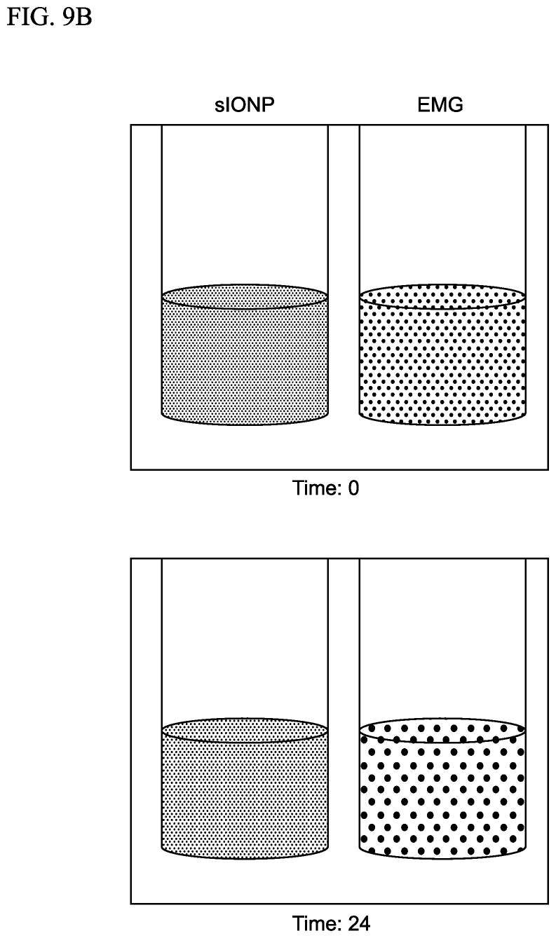

As shown in FIG. 8, IONPs embed in PHIL during precipitation rather than following solubility trends and transferring into aqueous solution. Both sIONPs and EMG are very soluble in water, but EMG is not colloidally stable in DMSO (see FIG. 9A) and EMG does not transfer from DMSO to water during precipitation and instead it is trapped by the PHIL precipitate. sIONPs, which are stable in both solutions, still embed in the PHIL precipitate rather than passing with DMSO into the surrounding water solution.

FIG. 9A is a graph showing DLS determination of stability of IONPs in DMSO. FIG. 9A shows a hydrodynamic radius of IONPs in DMSO alone, inset of percent of instrument counts. FIG. 9B is an image of the solutions of FIG. 9A at initial time and 24 hours. Aggregation and sedimentation (particles falling out of solution) occur with EMG308, but not with sIONPs.

### Example 13: MicroCT and MR Imaging of PHIL-IONP Tablets

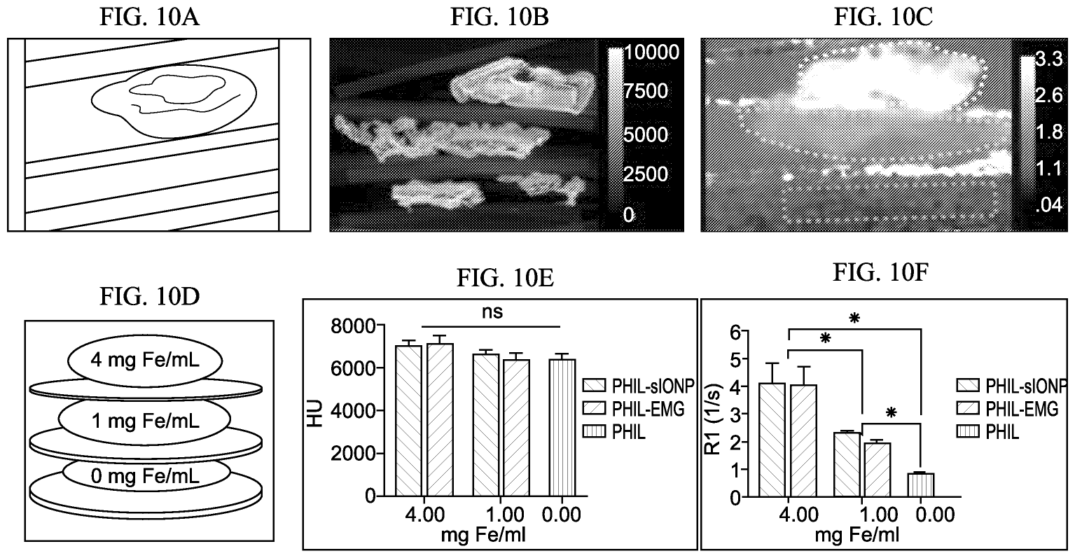

Image analysis of the microCT stacks of the PHIL and PHIL-sIONP tablets showed average HUs ranging from 6370-7130 HU (see E of FIG. 10). While this could locally vary from ˜1000-10000 HU in a given tablet, due to water pockets and porosity of tablets. Tablets' average HUs were not statistically different, regardless of Fe concentration meaning, there are no features in the microCT images (see FIG. 10B) one can use to differentiate between tablets. The samples are not significantly different by 1-way ANOVA which is not surprising since PHIL is designed to be radiopaque. While IONPs may also provide contrast on microCT, as there is a slightly increasing trend in HUs likely due to an increase in Fe density, they do not significantly affect PHIL's overall radio-opacity. In summary, microCT images of PHIL-sIONP did not show significant changes in HU compared to PHIL alone for different Fe concentrations.

Thus, one difference between PHIL and other embolics, such as Onyx and Squid, relates to its imaging properties on CT. As shown, even with the added IONPs, PHIL produces microCT images with high contrast and minimal artifacts (see FIG. 10B). In contrast, Onyx may suffer from streak artifacts (low HU and high HU streaks) on CT. Streak artifacts in general can lead to issues with determining the position of the embolic on CT and may interfere with detecting hemorrhage during a procedure. Squid, being the same as Onyx, but with smaller tantalum grain size, also suffers from streak artifacts on CT.

Onyx and Squid also share the characteristic that tantalum in both of these embolics sediments over time. Onyx, with a larger tantalum grain size, requires about 20 minutes of shaking prior to use and will settle over the course of a procedure. This leads to a decrease in radiopacity over time and therefore can make it difficult to determine embolic positioning. Squid, has smaller tantalum grain size, so to a lesser degree still settles over time. In comparison to Onyx and Squid, PHIL does not share the characteristic of settling of the radiopaque component iodine, as it is covalently bound to PHIL itself. The sIONPs do not settle and/or aggregate within the liquid embolic as seen with tantalum powder. Furthermore the IONPs do not visibly leach out of the embolic on precipitation (FIGS. 7 and 8).

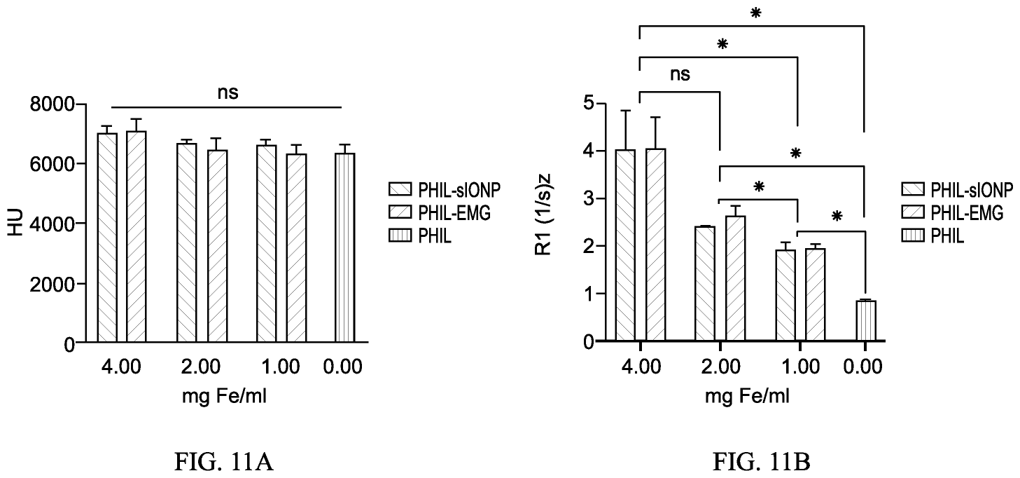

In contrast to microCT, MR imaging demonstrates contrast due to IONPs in PHIL. MR imaging allows for concentration of IONPs within the tablet to be determined from the R₁ map of a given PHIL tablet containing IONPs as well as other magnetic materials often result in largeR₁'s of the surrounding water, and therefore make standard R₁ measurements difficult or inaccurate. By using the MB-SWIFT sequence, images of large R₁ water signals can be obtained. Quantification of R₁ can be further improved by combining the MB-SWIFT with a Look-Locker acquisition scheme. PHIL-EMG and PHIL-sIONP both showed R₁ values that positively correlated to Fe concentration in the co-precipitate (see FIG. 10F) (EMG R₁ range=1.92-3.69 1/s, sIONP R₁ range=1.86-3.80 Us). There may be a slight increase in the R₁ of water (R₁-0.33 1/s) when PHIL is present (R₁=0.84+/−0.09 err 1/s) (see FIG. 10C), however PHIL by itself is not visually observable on MR imaging through T₁ mapping with Look-Locker MB-SWIFT or MB-SWIFT alone (see FIG. 10C). Statistical significance was completed by unpaired two-sample t-test between each set of Rivalues for a given concentration assuming equal variance between sample sets. FIG. 11A presents statistical analysis of μCT imaging using two-sample unpaired t-test (includes 2 mg Fe/mL PHIL-IONP tablets). FIG. 11B. presents statistical analysis of MR imaging using two-sample unpaired t-test (includes 2 mg Fe/mL PHIL-IONP tablets). ns denotes no significant difference, * denotes significance of P<0.05.

PHIL alone (0 mg Fe/mL) was statistically different from PHIL+4 mg Fe/mL IONPs (P=0.003) and PHIL+1 mg Fe/mL IONPs (P=0.00008). PHIL+1 mg Fe/mL IONPs was also statistically different from PHIL+4 mg Fe/mL IONPs (P=0.02).

While the R₁ values correlated well with the IONP concentrations in PHIL-IONP tablets, some changes in pixel locations due to the local magnetic field produced by IONPs, a.k.a. pileup effects, were observed at increased concentrations, 4 mg Fe/mL (see C of FIG. 2). Pileup may lead to position inaccuracies on the order of ˜1-2 mm.

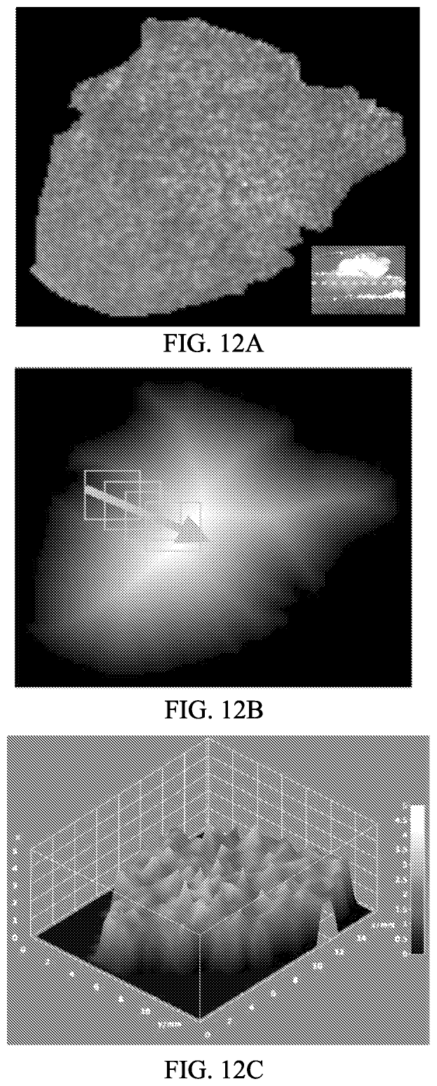

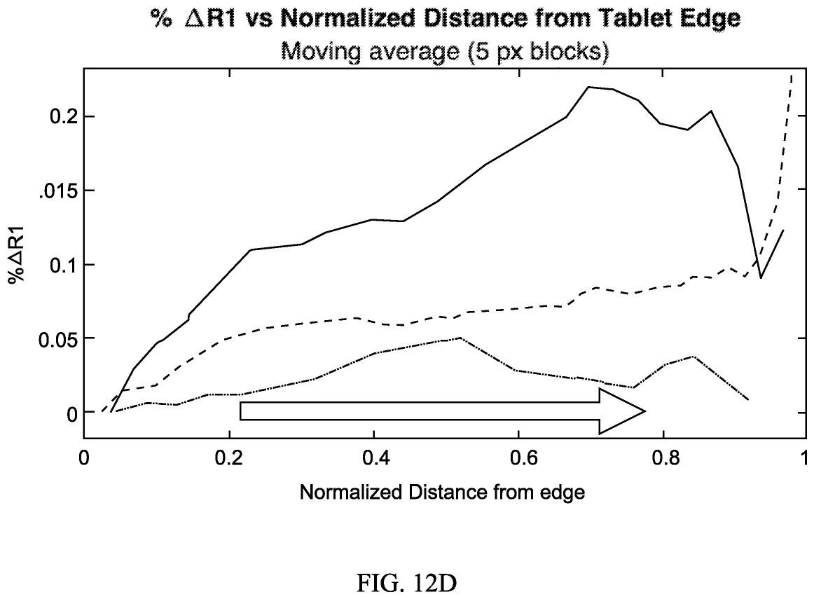

The IONPs may distribute unevenly within the embolic on precipitation due to shell formation trapping additional PHIL-IONP solution. This can be seen in FIGS. 12A-12D. FIGS. 12A-12D show a cross-section of the MR images of 1 mg Fe/mL tablets taken with the R1 as a function of distance away from the edge of the tablet is plotted for n=3 tablets at 1 mg Fe/mL in blocks of ˜0.6 mm as a moving average. Tablets at this concentration were specifically chosen as the pileup artifacts from the IONPs on MR imaging are lowest compared to the other two concentrations. The R₁ increases from the edge of the tablet to the center, indicating a higher concentration of IONPs in the center.

The MR imaging of PHIL alone also suggests minimal impact on T₁ and in comparison to Onyx and Squid (see FIG. 10C). Meanwhile, Onyx may appear hypointense (short T₁) on T₁-weighted imaging in MRI. Therefore, Fe quantification of IONPs in Onyx may not be possible using MRI. This is because this method of using SWIFT to detect IONPs is primarily T₁-weighted, with decreasing T₁ corresponding to increased IONP concentrations, so a further decrease in T₁ would potentially limit the upper bound of IONP quantification. Squid, since it is very similar to Onyx, may still produce variable T₁ hypointensities on MR imaging.

IONP concentration measurements on vessel like structures (tube like) could also yield more uniform R₁ values and therefore concentrations on imaging. In this case, the solution would precipitate immediately on contact with the surrounding water due to surface to volume ratio, minimizing inhomogeneities from IONP distribution during PHIL precipitation.

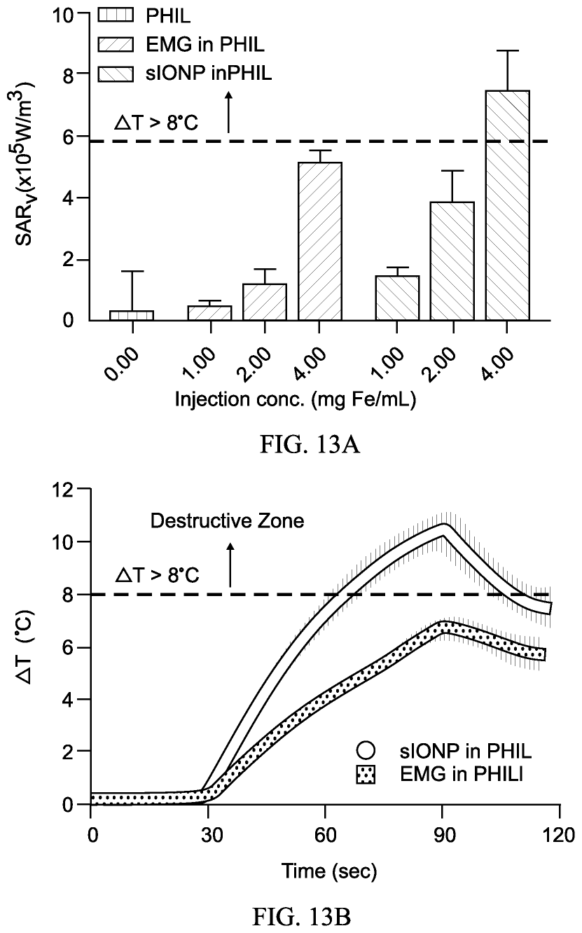

FIG. 13 is an example data set of heating of tablets in a 15 kW coil at 184 kHz and 65 kA/m. A of FIG. 13 shows the effect of varying concentration and type of IONPs on SAR_{V}. Higher concentration result in higher power deposition per unit volume (SAR_{V}), and sIONPs have higher SAR_{y} than EMG308. B of FIG. 13 shows temperature rise normalized (to initial temp) for 5 mg Fe/mL of sIONP and EMG308 co-precipitated in PHIL in water. Observation of 8° C. temperature rise above the initial temperature (23° C. in vitro for study and 37° C. in vivo) shows that the sIONPs should be able to potentially achieve temperatures capable of tumor hyperthermia (>45° C.) as opposed to EMG308. Dashed lines in A represents SAR_{V} estimated for a ΔT of 8° C. for time span of 60 sec. Dashed line in B represents ΔT threshold of 8° C. (i.e., minimum therapeutic temperature rise required).

Referring collectively to FIGS. 10A-10F, shown in a comparison of microCT and MR imaging of PHIL-sIONP tablets. FIG. 10A shows photos of PHIL-sIONP tablets in a 2 cm NMR tube. Tablets are layered in water with Teflon spacers at concentrations of 4, 1 and 0 mg Fe/mL. FIG. 10B shows representative microCT imaging of NMR tubes of PHIL precipitate with sIONPs (121 kV, 150 μA, 0.053 mm resolution). PHIL is a high CT contrast agent and IONPs marginally increase contrast. FIG. 10C shows MR imaging of PHIL precipitate with IONPs. This representative image was filtered via non-local means filter in MATLAB and despeckled in ImageJ to remove noise. PHIL is almost invisible. IONPs add contrast with intensity increasing with Fe concentration (16.4T, MB-SWIFT Look Locker sequence, 0.117 mm resolution). FIG. 10D shows a diagram of PHIL-IONP tablet positioning within tube. FIG. 10E shows a graph of Hounsfield units of IONPs (sIONP and EMG308) coprecipitated in PHIL tablets (ns denotes no significant difference). FIG. 10F shows a graph of R1 values of IONPs (sIONP and EMG308) coprecipitated in PHIL tablets (asterisk indicates P<0.05 via unpaired two-sample t-test).

### Example 14: Heating Capability of IONPs in PHIL Precipitate

IONPs embedded in PHIL precipitate are able to heat reproducibly and proportional to the amount of Fe. The heating of PHIL-EMG may be lower than PHIL-sIONP for the same concentrations as shown in FIG. 13A. This may be due to aggregation instability of EMG in the PHIL-DMSO precursor solution as shown in FIGS. 9A and 9B. Heating capability (SAR_{V}) which is related to the rate of heating (see SAR_{V} calculations) may be shown to increase for both IONPs as Fe concentration increased in PHIL. At 4 mg Fe/mL, sIONPs in PHIL are able to generate more than the minimum therapeutic temperature rise (ΔT) needed for magnetic hyperthermia, e.g., >8° C. above control (FIG. 13B), which in the body would lead to a hyperthermic (i.e. destructive) temperature, e.g., >45° C., if applied for an appropriate duration. PHIL-EMG was only able to reach 6.5° C. above initial temperatures under similar conditions.

For example, this temperature elevation of 8° C. above physiologic body temperature (37° C.) in a patient would allow a treatment temperature of 45°, which may cause significant cell necrosis when applied for 30 minutes (equivalent thermal dose to 43° C. for 120 min).

At increased temperatures, the time needed for necrosis is decreased exponentially, until 60° C. where necrosis is effectively instantaneous (seconds).

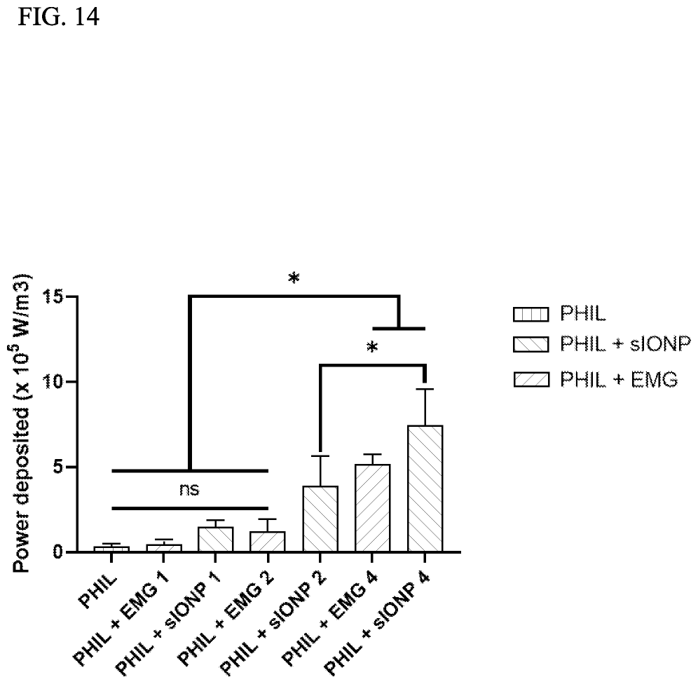

The concentration of IONPs in PHIL plays an important role in heating. For instance, as seen in A in FIG. 13, both PHIL-EMG and PHIL-sIONP have higher SAR_{V} (higher heating) with increasing concentrations compared to PHIL alone without any IONPs. FIG. 14 is a graph of a statistical analysis of tablet heating using one-way ANOVA. Ns denotes no significant difference, while an asterisk denotes significance of P<0.01.

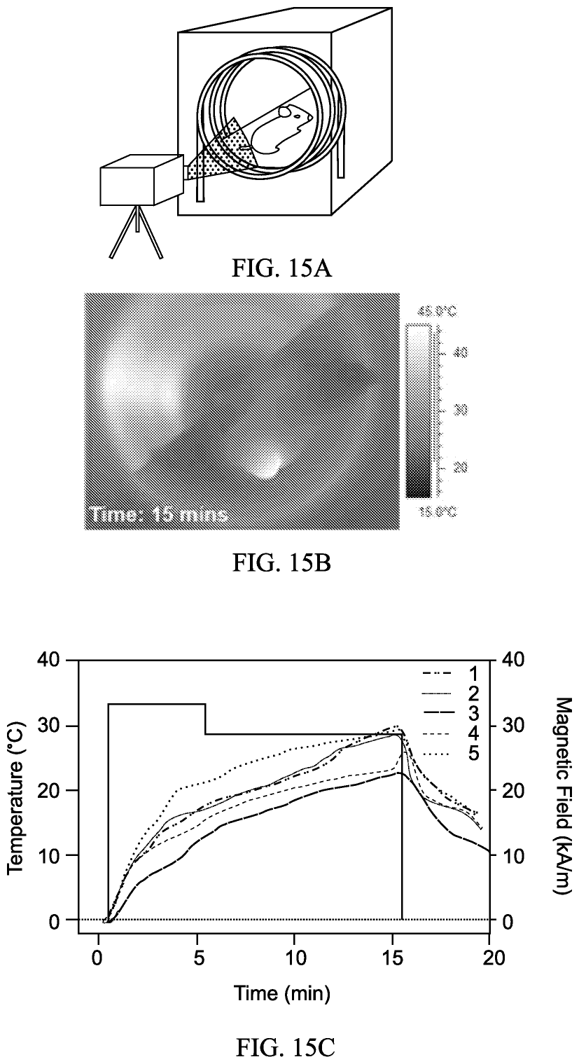

The differences in SAR_{V} for PHIL, PHIL-sIONP at 1 mg Fe/mL, PHIL-EMG at 1 mg Fe/mL or 2 mg Fe/mL are not statistically significant. Further increasing the concentration of IONPs increases the heating in both EMG & sIONP above the reference solution. As shown in B in FIG. 13, 4 mg Fe/mL had the highest heating in both IONPs although further increase in heating is possible by increases in concentration to at least 8 mg Fe/mL as shown in FIGS. 15A-15C.

Collectively, FIGS. 15A-15C are an example result demonstrated by the heating of mice post mortem in a 120 kW RF coil with 8 mg Fe/mL IONPs in PHIL injected in the hindlimb tumor. FIG. 15A is an illustration of the heating setup inside the RF coil. FIG. 15B is an IR image of post mortem mouse heating at 15 minutes for the 8 mg Fe/mL injection case. FIG. 15C is a plot of temperatures for mice injected with 0.2 mL of 8 mg Fe/mL sIONPs in PHIL DMSO solution in the coil at 365 kHz and 33.2 kA/m (94% coil power) for 0-5 minutes, 28.5 kA/m (80% coil power) for 5-15 minutes, and coil off after 15 minutes. The blue line is the magnetic field strength for the 8 mg Fe/mL heating trials.

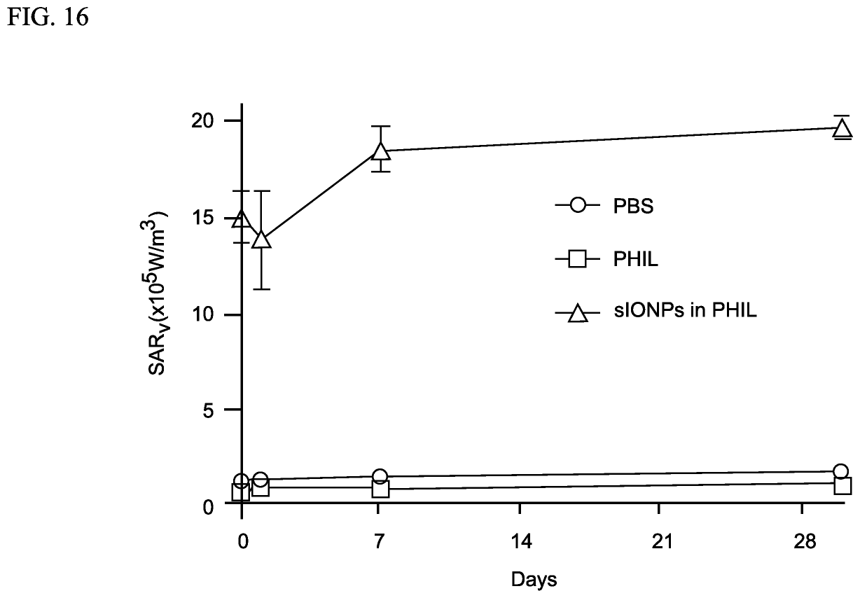

The extent of heating may also depend on the stability of the IONPs in solution. sIONPs are EMG coated in silica, both of which have a SAR_{Fe} of approximately 400 W/g Fe in water. While they share similar SAR_{Fe}, a major difference lies in their stability in solution. sIONPs are stable in DMSO, while EMG is not, as can be seen by hydrodynamic diameter measurements and photos in FIG. 9. As aggregation of IONPs affects the heating capability of these particles in solution, the same likely applies when entrapped in a precipitate. Thus, IONPs once aggregated in solution, like EMG in DMSO, will likely have lower heating after precipitation due to aggregation. sIONPs coprecipitated with PHIL have better heating than EMG coprecipitated with PHIL at comparable concentrations even though SAR_{Fe} are identical in aqueous suspension. Further, increasing the concentration of IONPs increases the heating for both EMG and sIONPs. As shown in B of FIG. 13, 4 mg Fe/mL concentration demonstrated the highest heating for both types of IONPs. Further increases in heating are possible by increasing concentration to 8 mg Fe/mL which is shown later in FIG. 16.

### Example 15: Colloidal Stability of IONPs Solutions

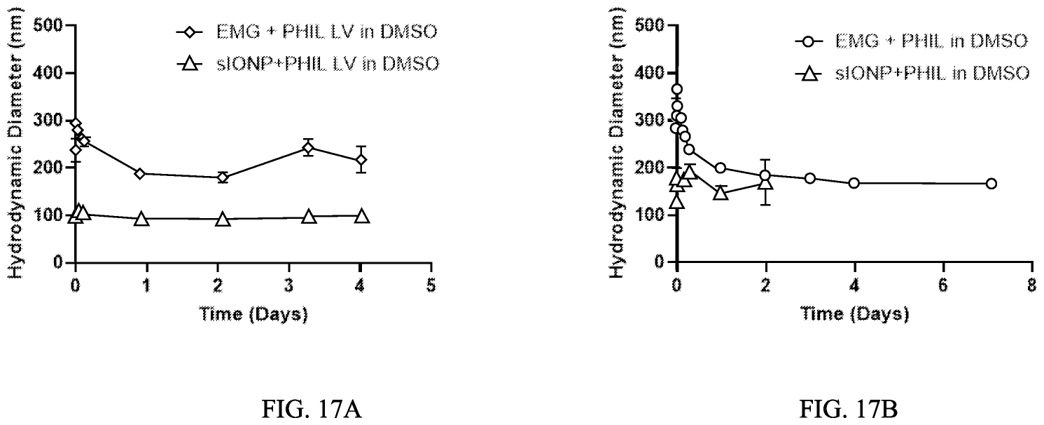

In order to retain maximum heating of IONPs in PHIL precipitate, IONPs need to be stable in the PHIL precursor solution, which contains DMSO. EMG and sIONPs were tested for colloidal stability in three solutions; DMSO (FIG. 9A), PHIL-DMSO (see FIG. 17B), and PHIL-LV-DMSO (PHIL-low viscosity a smaller MW of polymer) (see FIG. 17A). FIG. 9A is a graph of a DLS determination of stability of IONPs in DMSO solution. FIG. 17A is graph showing hydrodynamic radius of IONPs in PHIL-LV-DMSO. FIG. 17B is a graph showing hydrodynamic radius of IONPs in PHIL-DMSO.

The colloidal stability of each IONP in DMSO compared to that in PHIL-DMSO or PHIL-LV-DMSO was very similar. EMG is not stable in DMSO, whereas sIONPs, (silica-coated EMG), are stable in DMSO (visually seen in FIG. 9B) and the same is true in PHIL-DMSO or PHIL-LV-DMSO. The size of EMG aggregates was initially large, at 450 nm and slowly dropped to 250 nm after 10 days. As a comparison, the size of EMG particles in water is 45 nm. EMG may aggregate instantaneously, where the largest aggregates sediment quickly and smaller aggregates follow over several days. The size of EMG in PHIL-DMSO or PHIL-LV-DMSO matched this pattern and started above 500 nm. sIONPs were more stable in DMSO and PHIL-DMSO, both showing stable sIONP size with an average size of 100 nm. This matches well with a previous measurement of a 104 nm effective diameter of sIONPs in water. The size of sIONPs initially started at 120 nm and dropped to 115 nm in 6 hours. After 7 days it was still 115 nm. This initial drop is likely due to any large aggregates breaking apart or falling out of solution, where the remaining IONPs are stable. The size did not change significantly during the rest of the trial in PHIL-DMSO or PHIL-LV-DMSO. The stability of sIONPs on visual inspection is higher than that of EMG as seen in B of FIG. 9.

### Example 16: Heating Inside Agarose AVM Model

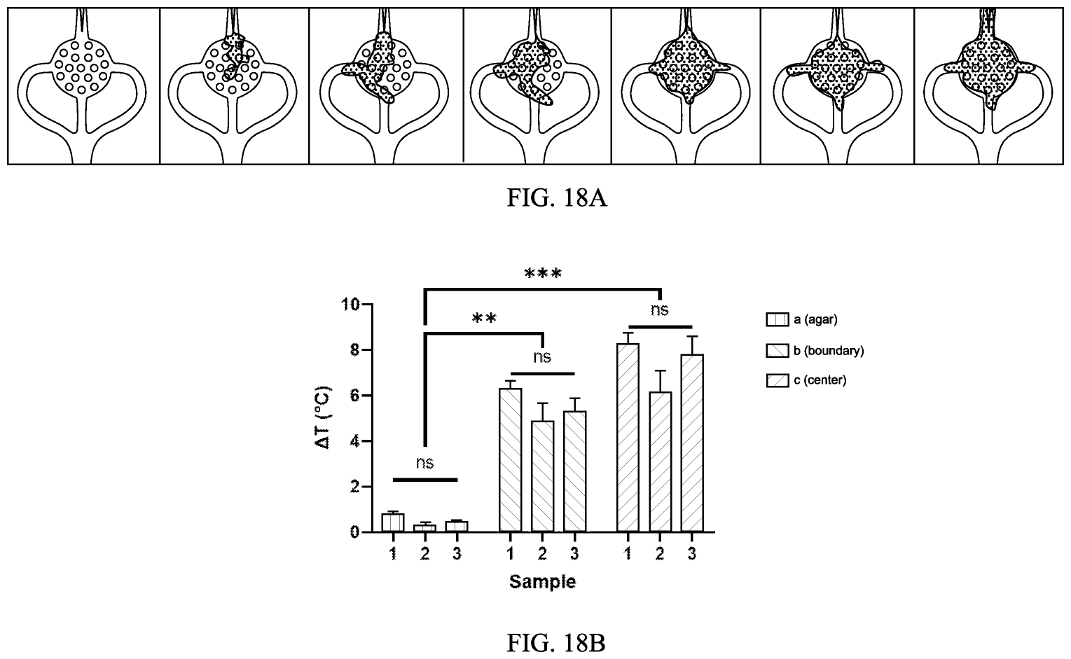

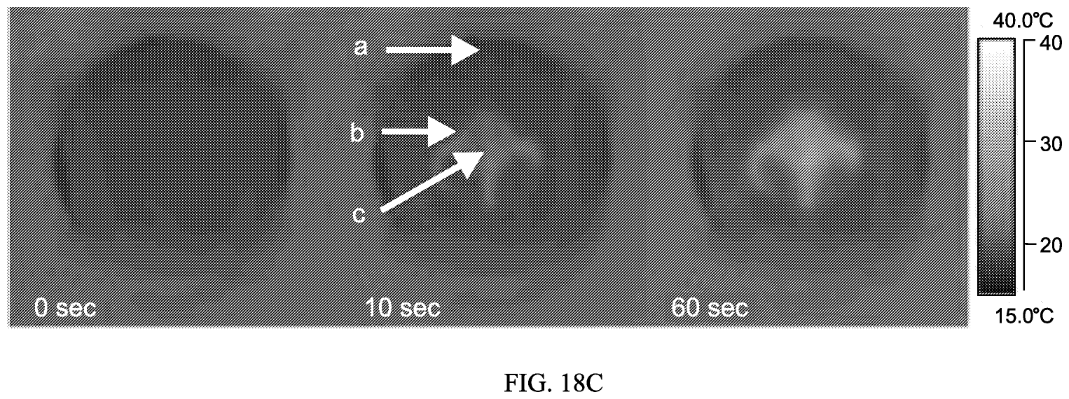

The deployment of PHIL may be seen in PHIL-sIONP coprecipitation in an in-vitro AVM model made from agarose gel from a previous silicone model (FIG. 4) under an RF field (FIGS. 18A-18C).

Collectively, FIGS. 18A-18C show an AVM model with a 4 mg Fe/mL PHIL-DMSO-sIONP injection and heating on 120 kW RF coil (as described above). FIG. 18A shows timelapse photos images of PHIL-sIONP co-precipitation in an agarose AVM model FIG. 4 (top, white model). Needle was placed in the model inlet and PHIL-DMSO-sIONP is injected into the center of the grid. Tan color is precipitated PHIL-sIONP and dark areas are where PHIL-sIONP is still in liquid phase, PHIL-sIONP virtually completely solidified after 20 min continuous flow of water. FIG. 18B is a graph showing temperature rise from initial temperature after 60 seconds of heating in RF coil (n=3 repeats) from three locations on AVM models face: a: agarose furthest from center, b: boundary of vascular bed, c: center of vascular bed. Statistical analysis of the AVM 3D model heating used one way Anova where “n”s denotes no significant difference, “**” denotes significance of P<0.001, and “***” denotes significance of P<0.0001. Additional statistical significance is shown between the center 1 and the boundaries of 2 and 3, and between the center of 3 and the boundary of 2. FIG. 18C shows an IR temperature image of the shower drain model at room temperature, up to 60 seconds.

Agarose gel is used as a simple physiological model for brain tissue and tumor models and is mostly water, which does not couple to RF fields significantly. 3% agarose was used to balance the firmness of higher concentrations of agarose and the flexibility seen at lower concentrations as the compression seals of the set up required mechanical strength under compression and flexibility for water seal. The agarose concentration is not expected to affect the overall understanding of precipitation or heating.

The PHIL-sIONP (4 mg Fe/mL) was injected into the center of the model's capillary bed grid and allowed to precipitate for 10 min under a continuous flow of water before heating in an RF coil (see FIG. 18A). PHIL is a white precipitate without IONPs, whereas the IONPs add a tan coloring to the precipitate and make the injection solution a dark brown color. The precipitate edges turn tan instantly, as seen at 20 seconds, on contact with water. The inner areas, where DMSO has not diffused away from the PHIL-IONPs, remains dark brown. As the DMSO diffuses out of the porous precipitate matrix, the polymer deposits and the area changes to a tan color. The IONPs are not visually seen flowing away from the precipitate at any point in time, as the surrounding solution in the output channels remains completely clear. DMSO and water are both colorless and transparent, but IONPs even at low concentrations (0.1 mg Fe/mL) in either solution are visible (FIGS. 7A and 7B). IONP precipitation with PHIL is not affected by the flow and shear of water through and around the forming precipitate. Once water flushes DMSO away, a porous structure is left, which can be back-filled with additional PHIL-IONP solution, as seen in FIG. 18A from 35 to 60 seconds. The concentration of IONPs can be increased, e.g., tripled, by slowly back filling space resulting from DMSO diffusion. Pausing the injection to allow DMSO to diffuse away, creates space for additional PHIL-IONPs to precipitate upon restarting injection. Backfilling porous areas results in both better embolization and increased concentrations of IONPs for increased heating.

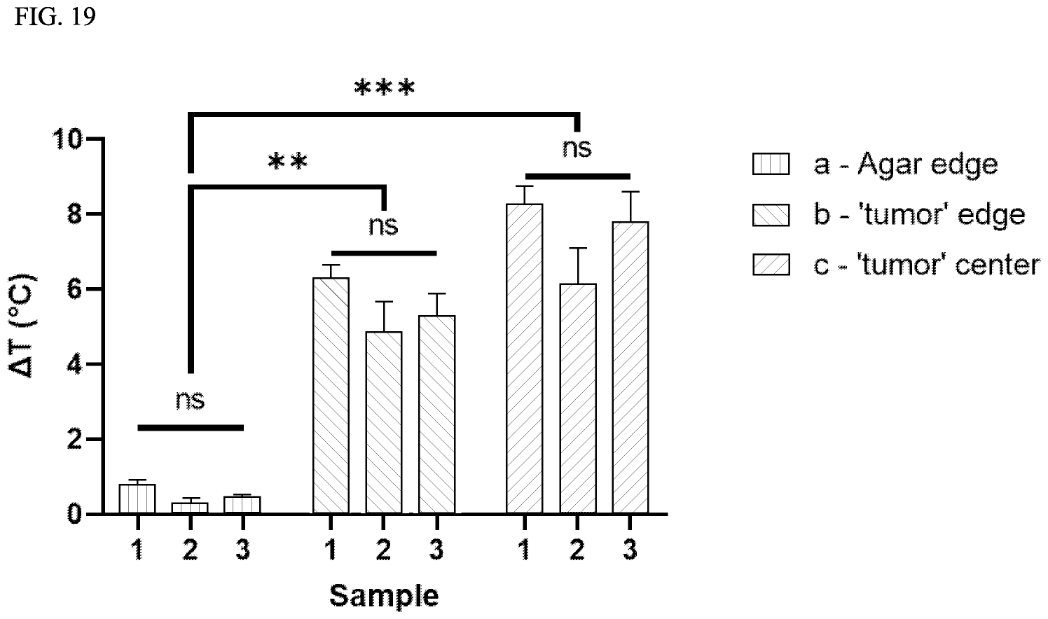

When injected into the agarose AVM model 4 mg Fe/mL sIONP with PHIL in DMSO was able to heat repeatably. Heating was monitored by an IR camera to compare the temperature increase of agarose to the AVM model grid with PHIL-IONP precipitate. The perimeter of the agarose (location a—FIG. 18B) was used as a convective heating control. The boundary edge of the grid (location b) was compared to the center of the grid (location c) to understand the uniformity of heating throughout the precipitate. Each sample has slightly different heating as the three embolic injections precipitate slightly differently. All precipitates were seen to have significant heating over the agarose, which did not increase significantly (see FIG. 18C). Statistical analysis may be done by one-way ANOVA as in FIG. 19. FIG. 19 is a graph of statistical analysis of the AVM 3D model heating of 8 mg/mL sIONPs in PHIL using one-way ANOVA. The location on the agarose model of each sample is denoted by letter and description. “ns” denotes no significant difference, “**” denotes significance of P<0.001, and “***” denotes significance of P<0.0001. Additional statistical significance is shown between the center of 1 and the edges of 2 and 3, and the center of 3 and the edge of 2. The center of the model increased in temperature 6-8° C. and the boundary of the grid reached 75% as high in the 60 seconds tested (see FIG. 19). Temperature increase was relatively consistent over each of the 3 trials on individual samples, suggesting PHIL precipitate is not being degraded or changed by increased temperature or heating gradients.

FIG. 16 is a graph of stability of heating in physiological conditions, 8 mg Fe/mL of sIONPs in PHIL in 1 mL PBS on an incubating shake at 37° C. and 170 RPM for 30 days. Heating time points taken on 1 kW Hotshot inductive heating systems with 2.75-turn, water-cooled copper coil (Ameritherm Inc., Scottsville, NY), 360 kHz and 20 kA/m. Heating is consistent over 1 month. Asterix denotes slight increase over time that is likely due to inward collapse of precipitate (which is not expected to occur when the embolic is embedded in the tissue) increasing local concentration of Fe around the fiber optic probe.

### Example 17: Heating Stability Under Physiological Conditions

To better show the stability of IONPs in a physiological setting, heating was tested on 8 mg Fe/mL of IONPs in PHIL at multiple time points over 1 month (FIG. 16). To simulate physiological conditions a PBS buffer was used to mimic the pH and osmolality of the human body. An incubating shaker was used to keep the temperature at 37° C. and moving, similar to blood flow. This test is important since IONPs in solution have been known to degrade or aggregate over time due to increased temperature, proteins or salt buffer. Nevertheless, IONPs in PHIL consistently heated with a SARᵥ˜15×10⁵ W/m³ on each test day suggesting repeated heating, shaking, PBS buffer, and time did not alter the reproducibility of heating.

The slight initial dip of the SAR_{V} (W/m³) is likely due to an initial shift in the location of the sIONP PHIL embolic mass away from the fiber optic insertion spot, or alternatively residual DMSO diffusing out of the precipitate over 1 day of shaking at 37° C. This second possibility would occur if there were significant (40-50% of injected DMSO) residual DMSO since it's heat capacity of ˜1.97 (J/g K) is less than ½ that of water's 4.18 (J/g K), which would imply that the temperature rise in DMSO would be larger (i.e. day 0) than water (i.e. day 1) under similar conditions. This is very unlikely due to the fact that DMSO diffusion from PHIL precipitate is fast and several rounds of 15 mLs of pure water were passed through the precipitate before initial testing.

The increase of heating between day 1 and 7 is attributable to the increase of PHIL at the center of the tube and therefore closer to the fluoroptic probe. The fluoroptic probes are removed during shaking and incubation periods, leaving an unsupported hole. The shaking at 170 RPM over the first week led to visual shifting of the PHIL super structure inwards. Without secondary support or networked shape, such as capillary beds, PHIL is freely moving cylinder inside the tube. Over time slight fractures formed, resulting in PHIL pieces shifting into the hole created for the fiber optic. The fiber optic was replaced on day 7 for the heating test into the same position, now overlapping with precipitate. This increase in PHIL and IONP density around the fiber optic results in an increase in temperature rise at that spot.

Notably, IONPs in PHIL precipitate does not decrease heating capability over time or after repeated testing. Thus, in principle a single PHIL-IONP injection could be used to heat a tumor multiple times over an extended period (PHIL has been shown to remain stable in vivo for months, as indicated in Fries et al., Treatment of Experimental Aneurysms with a GPX Embolic Agent Prototype: Preliminary Angiographic and Histological Results (J NeuroInvervent Surg. 2022)) allowing time to monitor tumor reduction, remission or relapse. Although other IONPs in embolic type agents such as hydrogels have been tested over 2 weeks, their heating saw significant decreases after 4 days, and complete loss after 2 weeks presumably due to IONP attrition from the gel.

### Example 18: Heating Capabilities in Tissue

To assess the ability of PHIL-IONP to heat tumors, the embolic was injected into mice with hind limb tumors postmortem and then heated repeatedly within an RF field. Ultimately, living animals will have blood flow surrounding the embolic site which may affect heating from IONPs and will need to be studied further. In this proof of principle example, postmortem animals were used with no blood flow to understand the capabilities of the 120 kW coil to heat for 15 minutes and customize the heating based on the concentration of IONPs in PHIL and magnetic field strength observed with an IR camera (see FIG. 15A). Injections of 0.2 mL of 8 mg Fe/mL IONPs in PHIL DMSO were used, as this volume has been commonly used with mice. Several initial tests were done at 0, 4, and 8 mg Fe/mL sIONPs in PHIL and various field strengths (FIG. 5). 33.2 kA/m (94% coil power) for 0-5 minutes and 28.5 kA/m (80% coil power) for 5-15 minutes coil settings were used to achieve the maximum stable field strength (i.e. highest initial heating) and then lowered to maintain a stable temperature increase (see FIG. 15C). Also, for larger scale treatments any amount of nonspecific heating (eddy heating) in healthy tissue could be avoided by lowering the field strength or frequency and increasing the IONP concentration to maintain the required heating (targeted therapeutic temperature). An average of 17° C. increase was seen after 5 minutes (see FIG. 15B), which, assuming minimal blood flow heat loss, would translate to 54° C. in-vivo, well into the therapeutic range for treatment. Heating reached a therapeutic levels, 8° C. after approximately 90 seconds, and remained above this for the entirety of the coil operation and an additional 5 minutes after the coil was turned off.

The depth of injection and spread of heat throughout the tumor and animal was observed by an IR camera. The temperature increase was found to be localized only to the injection site in the tumor, as seen in B of FIG. 5. This suggests that PHIL and therefore heat is localized to the tumor. It should be noted that there is a heating gradient similar to the agarose AVM model, which under sufficiently long heating of the tumor can exceed 8° C. at the tumor edge. Further refinement of sIONP PHIL heating can be tuned with IONP concentration changes, altered injection protocol (back filling), and visualization upon injection, through fluoroscopy.

Magnetic embolic heating of the PHIL-sIONP embolic is a method of treating vascularized tumors and can be performed multiple times with reproducible heating over 1 month in PBS at 37° C. Tumors can be injected with PHIL-sIONP and the embolized tumors can be heated in an RF coil to therapeutic temperatures. Additional injections of PHIL-IONP can be preformed to add additional heating to initial area of interest or secondary areas of interest. This embolic can be imaged on both μCT (largely PHIL contrast) and MRI using MB-SWIFT (principally IONP contrast), allowing for monitoring of embolic position after embolization, allowing for detailed treatment planning. Despite the ability to encapsulate both EMG (IONP core, Ferrotec) and silica coated EMG in PHIL, it is the sIONPs that are colloidally stable in DMSO and PHIL, allowing for higher heating rates to be generated at equivalent concentrations. The addition of IONPs to PHIL does not lead to premature precipitation of PHIL.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1-17. (canceled)

18. A method for thermally or hyperthermally treating an object, the method comprising:
preparing a precipitating hydrophobic injectable liquid (PHIL) embolic agent enhanced with a magnetic nanoparticle (NP);
advancing a delivery device to a target area;
injecting the PHIL-IONP embolic agent directly at the target area;
observing PHIL and IONPs in-situ using complementary imaging;
applying a first impulse to the target area at a first time to generate heat sufficient to thermally ablate or induce hyperthymia at the target area; and
applying secondary impulses to the target areas at a later time points to generate heat sufficient to ablate or induce hyperthymia at the target, wherein the additional times are subsequent to the first time.

19. The method of claim 18, wherein the PHIL embolic agent is radiopaque.

20. The method of claim 19, wherein the PHIL embolic agent is a nonadhesive copolymer and polyhydroxyethylmethacrylate (PHEMA) dissolved in DMSO with an iodine component covalently bound to the copolymer.

21. The method of claim 20, wherein the PHIL embolic agent is iodinated PLGA-PHEMA polymer.

22. The method of claim 18, wherein the magnetic NP comprises one or more of: an iron oxide nanoparticle (IONP), iron containing nanoparticle at various concentrations, doped iron oxide, and iron nitro nanoparticles.

23. The method of claim 22, wherein the magnetic NP remains embedded in the PHIL implant without diffusion or degradation throughout the duration of treatment.

24. The method of claim 18, wherein x-ray based imaging is used to locate the PHIL implant.

25. The method of claim 18, wherein magnetic resonance imaging (MRI) is used to quantify the distribution of the magnetic NP within the PHIL implant.

26. The method of claim 24, wherein the PHIL location and magnetic NP quantification can be used to quantify the distribution of expected heating and aid in treatment planning.

27. The method of claim 18, further comprising applying additional injection/s of PHIL-IONP embolic to target area for higher concentrated heating.

28. The method of claim 18, further comprising applying additional injection/s of PHIL-IONP embolic to secondary/adjoining area/s to increase area of heating, based on imaging

29. A system for thermally treating a tumor bed, the system comprising:
a first predetermined quantity of a precipitating hydrophobic injectable liquid (PHIL) embolic agent enhanced with:
a second predetermined quantity of an iron oxide nanoparticle (IONP); and
a delivery apparatus configured to provide a mixture of the IONP enhanced PHIL embolic agent directly to the tumor bed.

30. The system of claim 29, further comprising:
a first needle configured to deliver PHIL;
a second needle configured to deliver the mixture of magnetic nanoparticle (NP) enhanced PHIL embolic agent, wherein the PHIL delivered by the first needle can insulate the PHIL-magnetic NP and/or protect an area from heating.

31. The system of claim 30, wherein the magnetic NPs are IONP.

32. A system for observing PHIL-IONPs in situ, the system comprising:
an x-ray based imaging system;
a magnetic resonance imaging (MRI) system; and
a processor, wherein the x-ray based imaging system provides the location of the PHIL, wherein the MRI system provides quantitative information on the magnetic NP distribution within the PHIL implant, and wherein the processor is configured to combine the x-ray imaging and the MRI data to quantify the distribution of expected heating and aid in treatment planning.

33. The system of claim 32, wherein the magnetic NPs are IONP.

34. The system of claim 33, wherein combining the x-ray imaging and the MRI data to quantify the distribution of expected heating and aid in treatment planning comprises removing the MRI data from the x-ray imaging data to provide indication of the PHIL with the magnetic NPs.
